(19) United States
(12) Patent Application Publication
(10) Pub. No.: US 2016/0166791 A1
(43) Pub. Date: Jun. 16, 2016

(54) FLEXIBLE TUBE HOLDING DEVICE AND INTUBATION SYSTEM WITH METHODS OF USE

(71) Applicant: Bruce M. Kleene, Rochester, NY (US)

(72) Inventor: Bruce M. Kleene, Rochester, NY (US)

(21) Appl. No.: 14/568,142

(22) Filed: Dec. 12, 2014

(51) Int. Cl. A61M 16/04; A61B 1/04; A61B 1/247; A61B 1/00; A61B 1/267

(52) U.S. Cl. CPC A61M 16/0488; A61B 1/00071; A61B 1/00066; A61B 1/2673; A61B 1/247; A61B 1/2676; A61B 1/04; A61B 1/00165

(57) ABSTRACT

A flexible tube holding device includes a handle having a groove along a length of a back side thereof, the groove being sized to at least partially accept a desired flexible tube, such as an endotracheal tube. The device is generally J-shaped from the side, and further includes a blade connected to the handle, the blade having a proximal blade section connected to the handle and having an opening along a back side thereof, and a tube retention section connected to the proximal blade section at an end thereof opposite the handle and along the opening. The tube retention section is sized to accept and hold at least most of a cross-section of a tip portion of the flexible tube and shaped to retain the tip portion. The flexible tube is held in place manually or using a quick-release closure mechanism. The flexible tube holding device may be part of an intubation system. The system includes a flexible tube, an intermediate intubation device, such as a fiber optic bronchoscope, sized to fit within the flexible tube, and a flexible tube holding device. Methods of assembling, detaching and intubating using the flexible tube holding device and system are also provided.

### BACKGROUND OF THE INVENTION

1. Technical Field

The present invention generally relates to flexible tube holding devices and methods of use. More particularly, the present invention relates to intubation devices, systems and methods of use and intubation.

2. Background Information

Oral fiber optic endotracheal intubation is a well established airway management technique. It is accomplished in the following general manner. The operator inserts the tip of a fiber optic bronchoscope into the proximal end of the lumen of an endotracheal tube and advances the fiber optic bronchoscope until it is projecting out of the distal end of the endotracheal tube. The endotracheal tube may already have been positioned in the upper airway of the patient to be intubated. If this is not the case, the operator now positions the endotracheal tube/fiber optic bronchoscope complex into the upper airway of the patient. The operator seeks to acquire a view of the glottis by advancing or withdrawing the fiber optic bronchoscope and by using the control mechanisms built into the fiber optic bronchoscope. After acquiring such a view, the operator advances the tip of the fiber optic bronchoscope between the vocal cords and into the trachea. The operator then slides the endotracheal tube distally over the fiber optic bronchoscope and into the trachea. The fiber optic bronchoscope is removed, leaving the endotracheal tube in place.

It should be noted that there are airway instruments now available which are videoscopic rather than fiberoptic but which have the same general function as a fiber optic bronchoscope. For the sake of simplicity, in the following pages the term fiber optic bronchoscope will be used to refer to both fiber optic and similar videoscopic instruments.

Oral fiber optic endotracheal intubation is an important technique in the field of airway management. It is commonly used with some kinds of abnormal airway anatomy and when other methods of tracheal intubation have failed.

There are several disadvantages that limit the usefulness of oral fiber optic endotracheal intubation.

First, a view of the vocal cords may be difficult to acquire through the fiber optic bronchoscope. Soft tissues such as the tongue, tonsils, and abnormal masses may obstruct the view and interfere with attempts to direct of the distal end of the fiber optic bronchoscope. Fluids such as blood and saliva may interfere with the ability of the operator to see. Since most intubations are performed with the patient in the supine position, gravity causes secretions to pool in the posterior pharynx. Gravity also causes the path of the fiber optic bronchoscope to be along the posterior pharynx, increasing the likelihood that the secretions will obscure the distal end of the fiberoptic bronchoscope, making the vocal cords difficult or impossible to see.

Second, it is difficult to become skillful in the use of a fiber optic bronchoscope and to maintain that skill. The modern fiber optic bronchoscope is a sophisticated instrument not frequently in use by those who perform endotracheal intubations. Most anesthesia practitioners have been trained in its use but do not use it enough to maintain a high level of skill.

Third, the fiber optic bronchoscope is delicate instrument which is easily damaged and expensive to repair. The directional control mechanisms, consisting generally of thin cables running from the proximal end to the distal end of the instrument, are especially vulnerable to damage.

Oral fiber optic endotracheal intubation is a useful technique but one that is not reliably easy or quick. There are available a number of devices which serve to facilitate oral endotracheal intubation with a fiber optic bronchoscope, or which can be used for that purpose.

Intubating oral airways are one kind of device used for this purpose. These devices tend to direct the endotracheal tube along the back of the pharynx and have little functionality for protecting the tip of the fiber optic bronchoscope from mass effects and from secretions. They have little functionality with respect to soft tissue retraction and to the aiming of the fiber optic bronchoscope.

Devices incorporating tube guides can also be used for this purpose. Tube guides can exist on their own, as part of regular laryngoscopes, and as part of videoscopic laryngoscopes. These devices are relatively bulky, making them difficult to maneuver in the patient's upper airway, and thus diminishing their functionality for acquiring an image of the glottis, and for the aiming of endotracheal tubes with or without the presence of a fiber optic bronchoscope. The mechanism of separation of the devices from an endotracheal tube generally involves advancement of the endotracheal tube forward through the guide, sometimes with lateral displacement of the endotracheal tube out of the tube guide, followed sometimes by withdrawal of the device from around the endotracheal tube. The mechanism of separation thus is complicated, awkward, and potentially difficult. A known problem with such devices is that the advancement of the endotracheal tube through the device generates resistive friction that increases markedly with angulations approaching 90 degrees and with smaller radii of curvature.

There are also a variety of tubular or partially tubular devices which could serve this purpose. However, these are bulky and difficult to maneuver and have not gained clinical significance.

Thus, a need exists for instruments and their methods of use allowing for a simpler way to intubate using the general skills already possessed by those that regularly perform intubation.

### SUMMARY OF THE INVENTION

Briefly, the present invention satisfies the need for improved intubation instruments and procedures by providing a simplified flexible tube holding device and system with corresponding methods of use and intubation.

The present invention provides, in a first aspect, a flexible tube holding device. The flexible tube holding device includes a handle, the handle including a groove along a length of a back side thereof, and the groove being sized to at least partially accept a desired flexible tube. The device further includes a blade coupled to the handle, the blade including a proximal blade section coupled to the handle and having an opening along a back side thereof. The blade further includes a tube retention section coupled to the proximal blade section at an end thereof opposite the handle and along the opening, the tube retention section being sized to accept at least most of a cross-section of a tip portion of the flexible tube and shaped to retain the tip portion.

The present invention provides, in a second aspect, an intubation system. The system includes a flexible tube, an intermediate intubation device sized to fit within the flexible tube, and a flexible tube holding device. The holding device includes a handle, the handle including a groove along a length of a back side thereof, and the groove being sized to at least partially accept the flexible tube. The device further includes a blade coupled to the handle, the blade including a proximal blade section coupled to the handle and having an opening along a back side thereof. The blade further includes a tube retention section coupled to the proximal blade section at an end thereof opposite the handle and along the opening, the tube retention section being sized to accept at least most of a cross-section of a tip portion of the flexible tube and shaped to retain the tip portion.

The present invention provides, in a third aspect, a method of assembling an intubation system. The method includes providing a flexible tube, and a flexible tube holding device. The holding device includes a handle, the handle including a groove along a length of a back side thereof, and the groove being sized to at least partially accept the flexible tube. The holding device further includes a blade coupled to the handle, the blade including a proximal blade section coupled to the handle and having an opening along a back side thereof, and a tube retention section coupled to the proximal blade section at an end thereof opposite the handle and along the opening, the tube retention section being sized to accept at least most of a cross-section of a tip portion of the flexible tube and shaped to retain the tip portion. The method further includes inserting the tip portion of the flexible tube into the tube retention section of the device, bending the flexible tube into the groove of the handle, causing the flexible tube to be detachably held in the groove and the opening, and inserting an intermediate intubation device into a proximal end of the flexible tube and advancing it at least partly into the flexible tube.

The present invention provides, in a fourth aspect, a method of detaching a flexible tube from a flexible tube holding device. The method includes providing a system for intubation, the system including a flexible tube, and a flexible tube holding device, the flexible tube being detachably held against a back side of the flexible tube holding device. The flexible tube holding device includes a handle, and a blade coupled to the handle, the blade including a tube retention section sized to accept at least most of a cross-section of a tip portion of the flexible tube and shaped to retain the tip portion. The method further includes releasing the flexible tube from the back side of the flexible tube holding device, and pulling on a proximal end of the flexible tube to cause the flexible tube to slide proximally out of the tube retention section.

The present invention provides, in a fifth aspect, a method of intubating a patient. The method includes providing an intubation system, the system including a first flexible tube, and a flexible tube holding device. The flexible tube holding device includes a handle, and a blade coupled to the handle. The blade includes a tube retention section sized to accept at least most of a cross-section of a tip portion of the first flexible tube and shaped to retain the tip portion, the first flexible tube being detachably held against a back side of the intubating assisting device, and an intermediate intubation device. The method further includes inserting the intubation system into an upper airway of the patient and maneuvering the intubation system to expand the upper airway, visualizing the patient's vocal cords, advancing a distal end of the intermediate intubation device between the vocal cords and into the patient's trachea based on the visualizing, and releasing the first flexible tube from the handle of the flexible tube holding device. The method further includes pulling on a proximal end of the first flexible tube to cause the first flexible tube to slide proximally out of the tube retention section while minimizing movement of the intermediate intubation device, sliding a second flexible tube over the intermediate intubation device and into the trachea, removing the flexible tube holding device, and leaving the second flexible tube within the trachea of the patient.

These, and other objects, features and advantages of this invention will become apparent from the following detailed description of the various aspects of the invention taken in conjunction with the accompanying drawings.

### BRIEF DESCRIPTION OF THE DRAWINGS

FIG. 1 depicts one example of a flexible tube holding device, in accordance with one or more aspects of the present invention.

FIG. 2 depicts one example of an intubation system incorporating the flexible tube holding device of FIG. 1, in accordance with one or more aspects of the present invention.

FIG. 3 is a cross-sectional view of one example of the proximal blade section of the flexible tube holding device of the intubation system of FIG. 2, in accordance with one or more aspects of the present invention.

FIG. 4 is a cross-sectional view of one example of the tube-retention section of the flexible tube holding device of the intubation system of FIG. 2, in accordance with one or more aspects of the present invention.

FIGS. 5-8 depict one example of assembling the intubation system of FIG. 2, in accordance with one or more aspects of the present invention.

FIGS. 9-11 depict one example of detaching a flexible tube from the flexible tube holding device of FIG. 1, in accordance with one or more aspects of the present invention.

FIGS. 12-18 depict one example of a method of intubating a patient using an intubation system similar to that of FIG. 2, but using a different intermediate intubation device, in accordance with one or more aspects of the present invention.

FIGS. 19 and 20 depict one example of the flexible tube holding device of FIG. 1 with a flexible tube therein, and including one example of a quick-release closure mechanism, in accordance with one or more aspects of the present invention.

FIG. 21 depicts one example of an intubation system similar to that of FIG. 2, but using an elongated imaging device as the intermediate intubation device, in accordance with one or more aspects of the present invention.

FIGS. 22-29 depict one example of the intubating method of FIGS. 12-18 using the elongated imaging device of FIG. 32, in accordance with one or more aspects of the present invention.

FIGS. 30 and 31 depict examples of visualizing vocal cords of the patient using an elongated imaging device not contained within the first flexible tube (FIG. 30), and indirect visualizing by reflection (FIG. 31), in accordance with one or more aspects of the present invention.

FIG. 32 depicts a simplified example of an elongated imaging device, in this case a fiber optic bronchoscope, along with examples of other intermediate intubation devices, including an intubating bougie, an exchange catheter, a hollow intubating catheter, and an elongated intubation device within a hollow intubating catheter, the hollow intubating catheter being sized to accept the elongated imaging device, in accordance with one or more aspects of the present invention.

FIG. 33 depicts one alternate example of a flexible tube holding device similar to that of FIG. 1 except that the proximal blade section includes multiple different cross-sections, in accordance with one or more aspects of the present invention.

FIG. 34 depicts an example of the flexible tube holding device of FIG. 1 having a flat proximal blade section, in accordance with one or more aspects of the present invention.

FIGS. 35-38 depict steps in the intubation method of FIGS. 12-18 in which the intermediate intubation device includes an elongated imaging device, similar to the example of FIG. 22, but including a fiber optic bronchoscope inside a hollow intubation catheter, in accordance with one or more aspects of the present invention.

FIG. 39 depicts another example of a flexible tube holding device of the present invention being inserted into a patient, in accordance with one or more aspects of the present invention.

### DETAILED DESCRIPTION OF THE INVENTION

Aspects of the present invention and certain features, advantages, and details thereof, are explained more fully below with reference to the non-limiting examples illustrated in the accompanying drawings. Descriptions of well-known materials, fabrication tools, processing techniques, etc., are omitted so as not to unnecessarily obscure the invention in detail. It should be understood, however, that the detailed description and the specific examples, while indicating aspects of the invention, are given by way of illustration only, and are not by way of limitation. Various substitutions, modifications, additions, and/or arrangements, within the spirit and/or scope of the underlying inventive concepts will be apparent to those skilled in the art from this disclosure.

Approximating language, as used herein throughout the specification and claims, may be applied to modify any quantitative representation that could permissibly vary without resulting in a change in the basic function to which it is related. Accordingly, a value modified by a term or terms, such as “about,” is not limited to the precise value specified. In some instances, the approximating language may correspond to the precision of an instrument for measuring the value.

The terminology used herein is for the purpose of describing particular examples only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include (and any form of include, such as “includes” and “including”), and “contain” (and any form of contain, such as “contains” and “containing”) are open-ended linking verbs. As a result, a method or device that “comprises,” “has,” “includes” or “contains” one or more steps or elements possesses those one or more steps or elements, but is not limited to possessing only those one or more steps or elements. Likewise, a step of a method or an element of a device that “comprises,” “has,” “includes” or “contains” one or more features possesses those one or more features, but is not limited to possessing only those one or more features. Furthermore, a device or structure that is configured in a certain way is configured in at least that way, but may also be configured in ways that are not listed.

As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances the modified term may sometimes not be appropriate, capable or suitable. For example, in some circumstances, an event or capacity can be expected, while in other circumstances the event or capacity cannot occur—this distinction is captured by the terms “may” and “may be.”

As used herein, the term “connected,” when used to refer to two physical elements, means a direct connection between the two physical elements. The term “coupled,” however, can mean a direct connection or a connection through one or more intermediary elements.

Reference is made below to the drawings, which are not drawn to scale for ease of understanding, wherein the same reference numbers are used throughout different figures to designate the same or similar components.

The fiber optic bronchoscope mentioned previously is only one of a number of intermediate intubation devices. Intermediate intubation devices are elongated, flexible devices used by operators. In addition to fiber optic bronchoscopes and similar instruments of a videoscopic nature, the general category of intermediate intubation devices include a number of devices, of which the intubating bougie is typical. An intubating bougie is elongated, flexible, generally made of plastic, sized to fit within the lumen of a selected endotracheal tube, lacks a handle, and is typically incapable of acquiring, transmitting, or displaying images. Other similar intermediate intubation devices include endotracheal exchange catheters and hollow intubation catheters, such as the Aintree Catheter. Intermediate intubation devices are inserted distally by the operator through the glottis and into the trachea. The operator advances an endotracheal tube over the shaft of the intermediate intubation device and into the trachea, using the intermediate intubation device as an internal guide for passing the endotracheal tube. As will be shown below, the present invention is designed for use with the general category of intubation devices, which encompasses intermediate intubation devices.

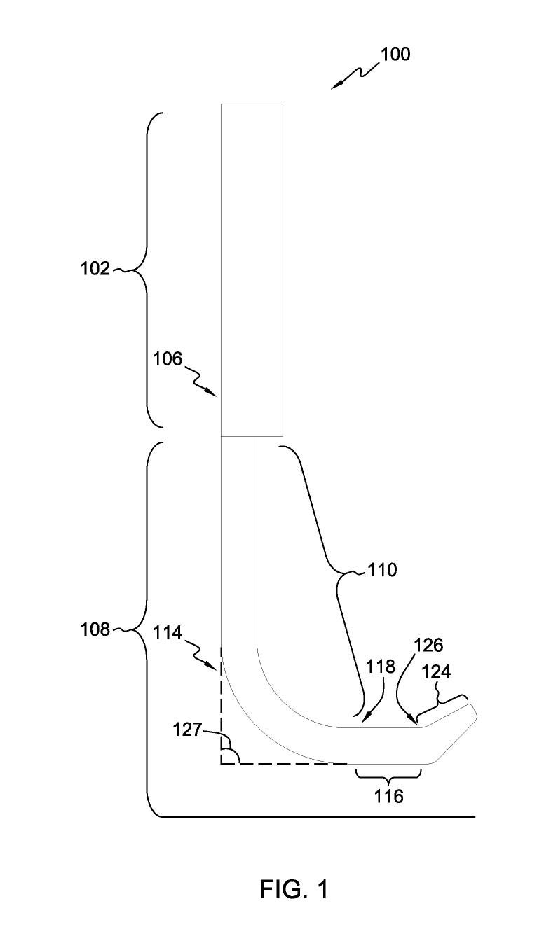

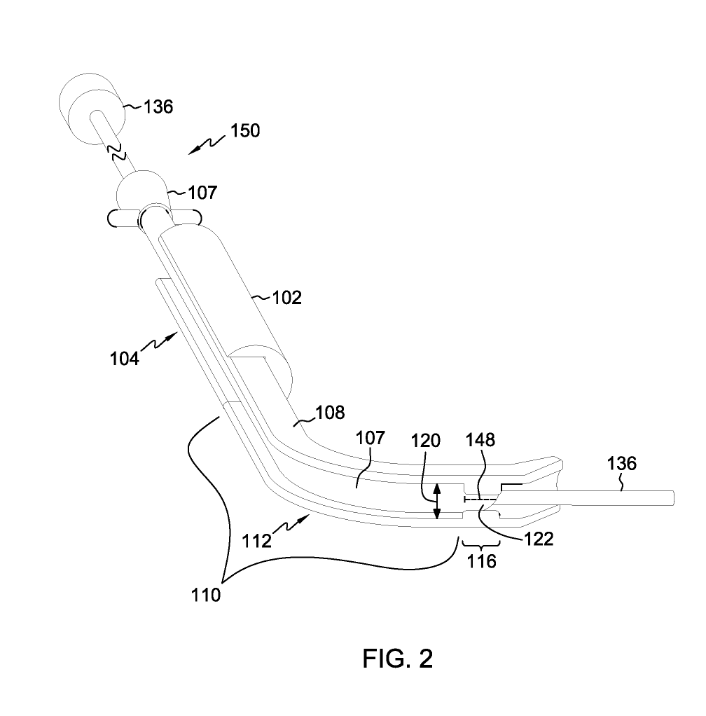

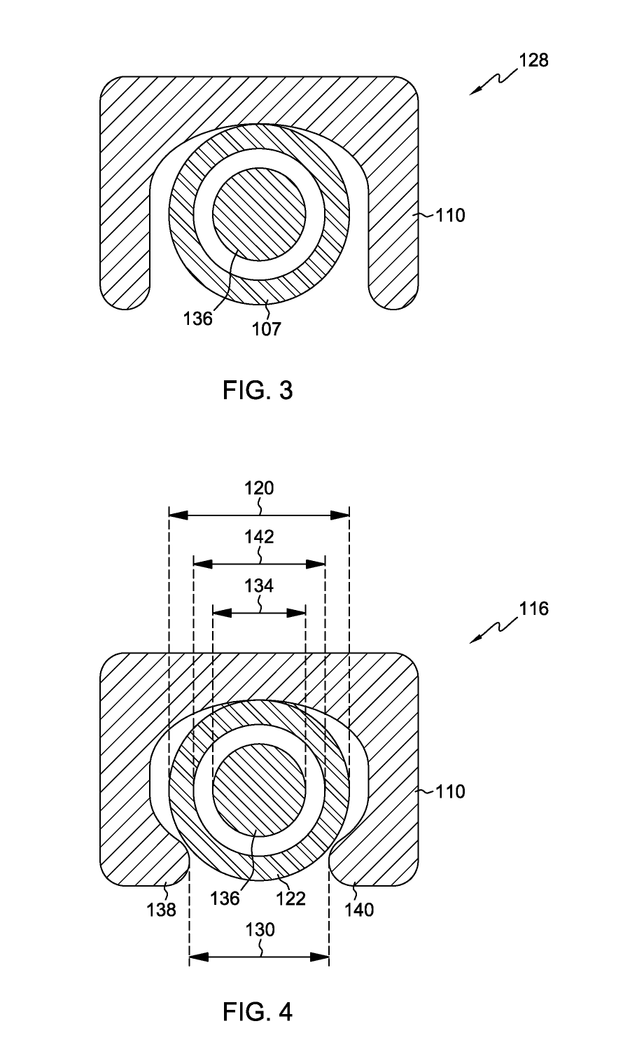

FIG. 1 depicts one example of a flexible tube holding device 100. The device includes a handle 102, the handle including a groove (104, FIG. 2) along a length of a back side 106 thereof, and sized to at least partially accept a desired flexible tube 107 (see FIG. 2), e.g., a standard size endotracheal tube. The device also includes and a blade 108 coupled to the handle, the blade including a proximal blade section 110 coupled to the handle and having an opening (112, FIG. 2) along a back side 114 thereof. The device further includes a tube retention section 116 coupled to the proximal blade section at an end 118 thereof opposite the handle and along the opening. The tube retention section may be sized to accept at least most of a cross-section (120, FIG. 2) of a tip portion (122, FIG. 2) of the flexible tube and may be shaped (see FIGS. 2 and 4) to retain the tip portion.

Continuing with FIG. 1, the device may further include a tip section 124 coupled to the tube retention section at an end 126 thereof opposite the proximal blade section 110. The tip section may have an upward tilt (best shown in FIG. 1). Further, as shown in FIG. 1, the blade preferably has a generally J shape, and may curve to an angle 127 of about 90° with respect to the handle.

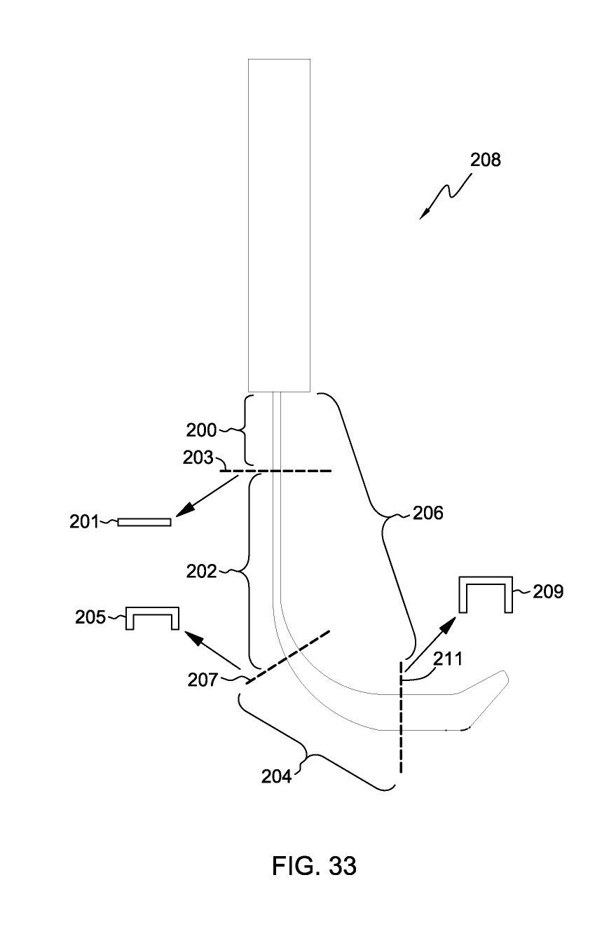

In one example, as shown in FIG. 3, a cross-section 128 of the proximal blade section 110 is preferably curved (see FIGS. 2 and 3). In one example, the cross-section may have a generally U or C shape and/or, as shown in FIG. 33, may include a plurality of subsections of different sizes and/or cross-sections. For example, subsection 200 of proximal blade section 206 of flexible tube holding device 208 has a flat cross-section 201 taken across line 203. Similarly, each of subsections 202 and 204 have different depth square U-shaped cross-sections 205 and 209, taken across lines 207 and 211, respectively.

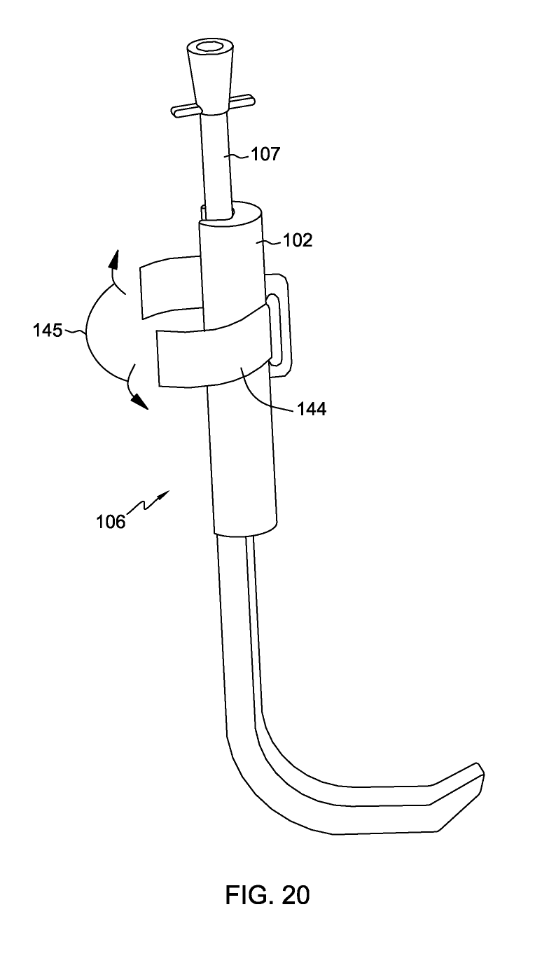

In another example, as shown in FIGS. 19 and 20, the flexible tube holding device may include a quick-release closure mechanism 144 coupled to the handle 102 to hold a flexible tube 107 in the groove (104, FIG. 2). The flexible tube 107 is preferably held to the back side 106 of the handle by the quick-release closure mechanism, shown in the open 145 (FIG. 20) and closed (FIG. 19) positions. In one example, the quick-release closure mechanism comprises a strap with hook-and-loop type closure.

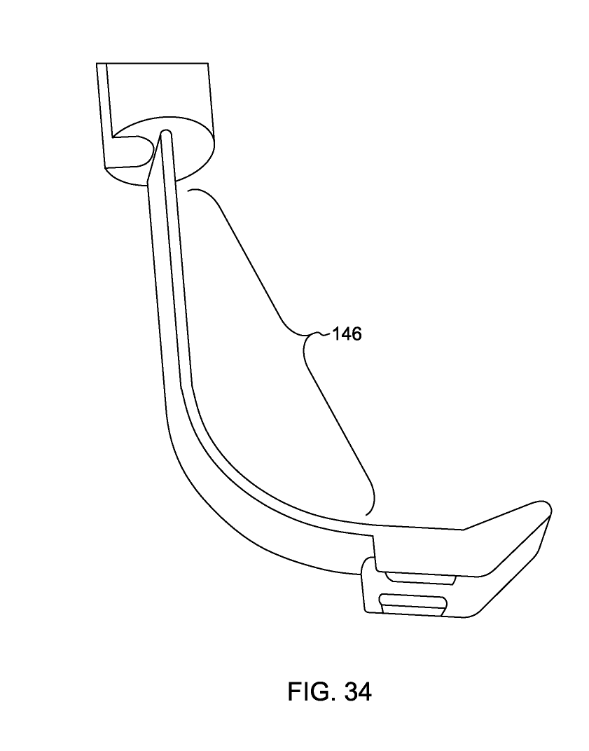

In another example, as shown in FIG. 34, the proximal blade section 146 may have a generally flat shape.

FIG. 2 depicts one example of an intubation system 150, according to one or more aspects of the present invention. The system includes a flexible tube 107, an intermediate intubation device 136 (e.g., a fiber optic bronchoscope), preferably sized to fit within the flexible tube, and a flexible tube holding device (100, FIG. 1). The flexible tube holding device includes handle 102 and a blade 108 coupled to the handle. The handle may include a groove 104 along a length of its back side (106, FIG. 1), and may be sized to at least partially accept the desired flexible tube. The blade includes a proximal blade section 110 coupled to the handle and having an opening 112 along the back side thereof (114, FIG. 1). The blade further includes a tube retention section 116 coupled to the proximal blade section at an end 118 thereof (see FIG. 1), opposite the handle and along the opening 112. Further, the tube retention section may be sized to accept at least most of a cross-section 120 of a tip portion 122 of the flexible tube, and may be shaped to retain the tip portion. Further, the tube retention section may be sized to hold no more than about 4 centimeters of a length 148 of the tip portion of the flexible tube.

Continuing with FIG. 2, the tube retention section 116 of the system 150 may further include a tip section (124, FIG. 1) coupled to an end of the tube retention section (126, FIG. 1) opposite the proximal blade section. Preferably, the tip section is designed to minimize tissue trauma, for example, using rounded elements, and may further include an upward tilt (see FIG. 1). Further, the blade preferably has a generally J shape, and may curve to an angle (127, FIG. 1) of about 90° with respect to the handle.

In one example, as depicted in FIG. 4, the tube retention section 116 includes a retention gap 130 of less than an outside diameter 120 of the flexible tube 107 (here, the tip portion 122, FIG. 2) and greater than an outside diameter 134 of the intermediate intubation device 136. The retention gap may be formed by at least two flanges 138, 140 extending inward from opposite sides of the blade (108, FIG. 1). In one example, the flanges are preferably substantially rigid. Further, the retention gap may be sized approximately equal to an internal diameter 142 of the flexible tube.

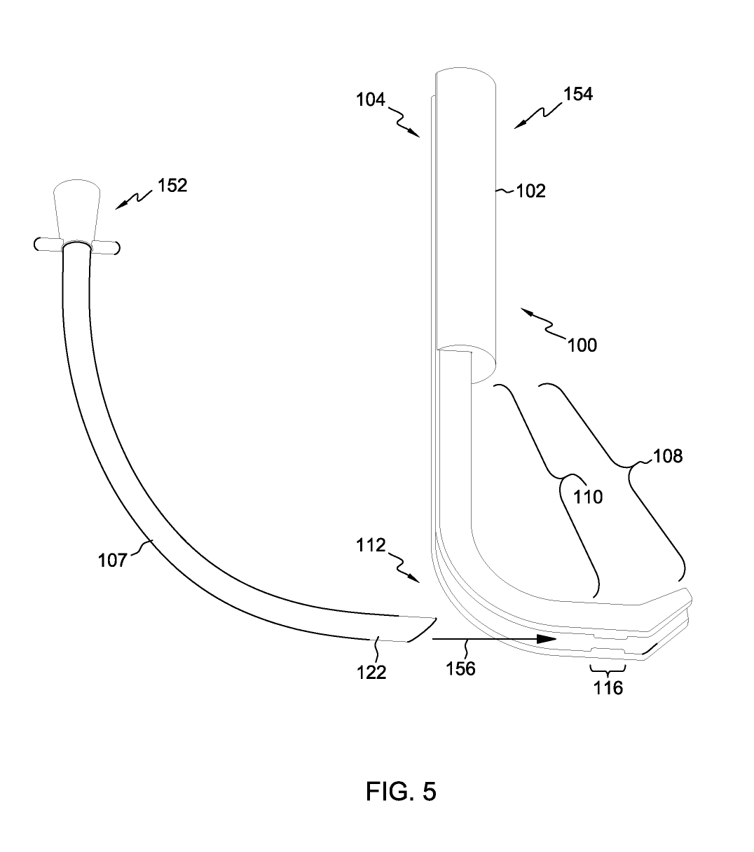

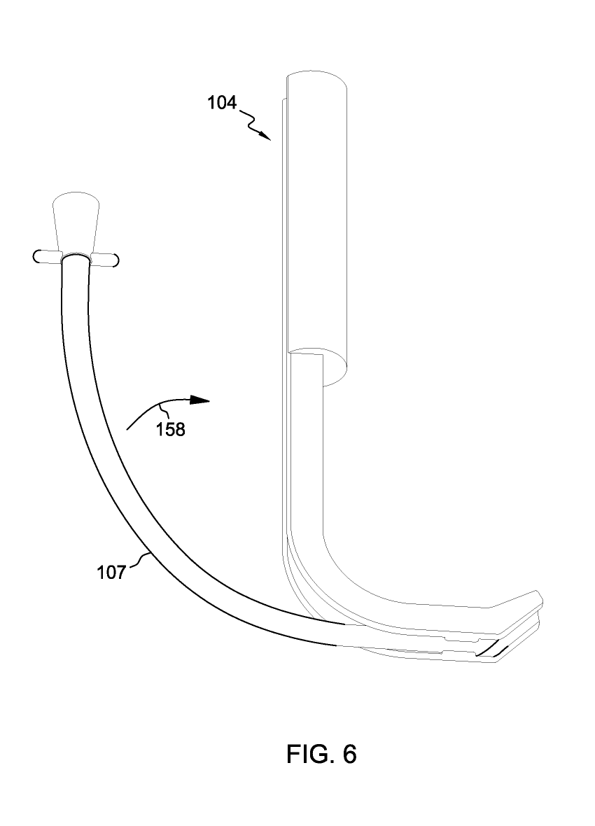

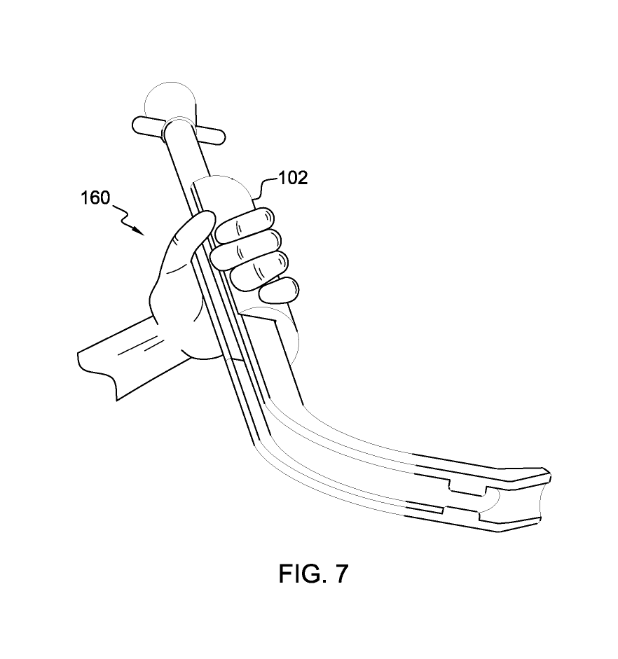

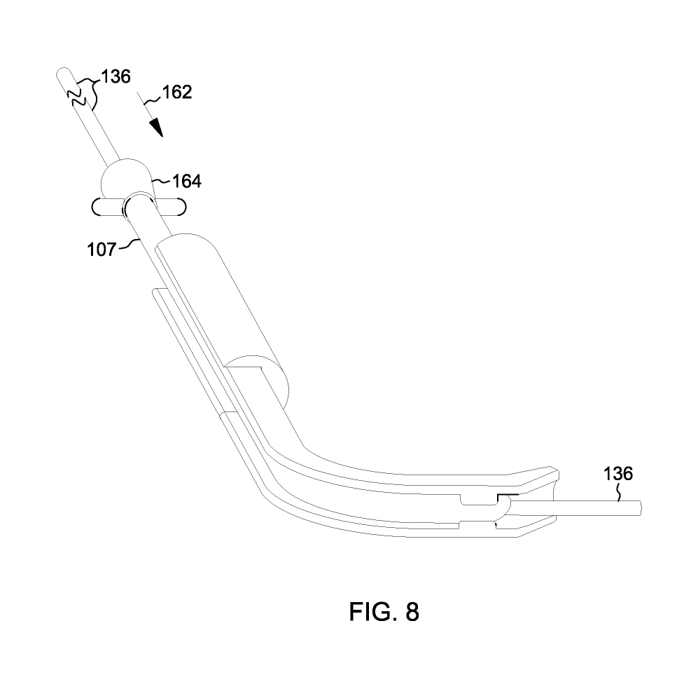

In one aspect of the invention, a method of assembling an intubation system is provided and described with reference to FIGS. 5-8. The method of assembling includes providing (152, FIG. 5) a flexible tube 107, and providing (154, FIG. 5) a flexible tube holding device (100, FIG. 5). The holding device includes a handle 102, the handle having a groove 104, on a back side (106, FIG. 1) thereof, and a blade (108, FIG. 5) coupled to the handle. The blade includes a proximal blade section (110, FIG. 5) coupled to the handle, and a tube retention section (116, FIG. 5) coupled to the proximal blade section at an end (118, FIG. 1) thereof opposite the handle and along the opening. The handle is sized to at least partially accept a desired flexible tube 107. Further, the proximal blade section, which may be curved, includes an opening (112, FIG. 5) along the back side (114, FIG. 1) thereof. In one example, the tube retention section may be sized to accept at least most of a cross-section (120, FIGS. 2 and 4) of a tip portion (122, FIG. 5) of the flexible tube and may be shaped to retain the tip portion, using friction from the sizing difference. Although not shown, a conventional inflated endotracheal tube cuff, or similar, may additionally be used to hold the flexible tube in place. The method further includes inserting (156, FIG. 5) the tip portion of the flexible tube into the tube retention section of the device, bending (158, FIG. 6) the flexible tube into the groove of the handle, causing the flexible tube to be detachably held (e g , manually held 160, FIG. 7) in the groove and in the opening (112, FIG. 5) of the proximal blade section (110, FIG. 5), and inserting (162, FIG. 8) an intermediate intubation device (136, FIG. 8) into a proximal end 164 of the flexible tube and advancing it at least partly into the flexible tube.

As noted above, in one example, as shown in FIG. 7, causing the flexible tube to be detachably held may include manually holding the flexible tube in the groove of the handle. In another example, as described above with respect to FIGS. 19 and 20, the device may include a quick release closure mechanism 144 coupled to the handle to hold the flexible tube in the groove at the back side of the handle, and causing the flexible tube to be detachably held may include closing the quick-release closure (FIG. 20).

Further, the tube retention section (116, FIG. 5) may include a retention gap (130, FIG. 4) of less than an external diameter (120, FIG. 4) of the flexible tube and greater than an external diameter (134, FIG. 4) of a desired intermediate intubation device (136, FIG. 4), and may be sized (116, FIG. 2) to accept no more than about 4 centimeters of the length (148, FIG. 2) of the tip portion of the flexible tube.

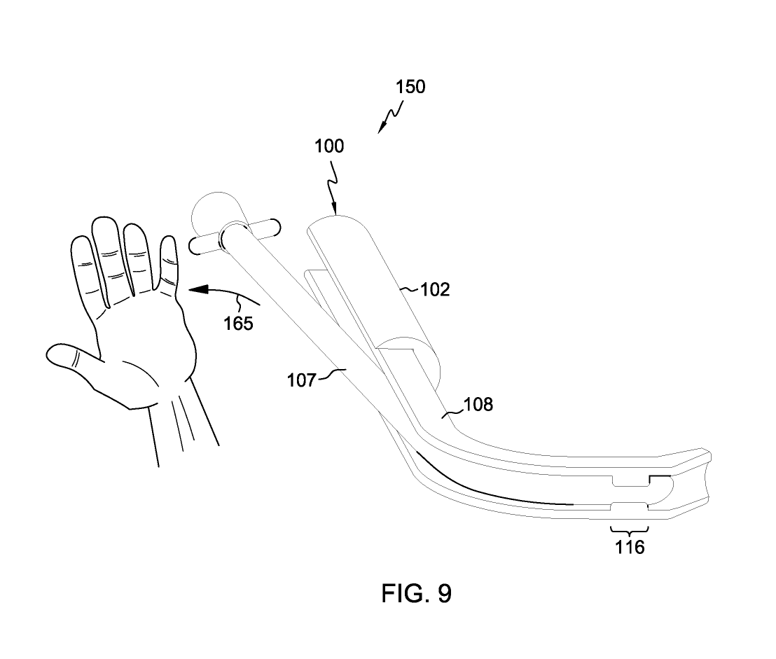

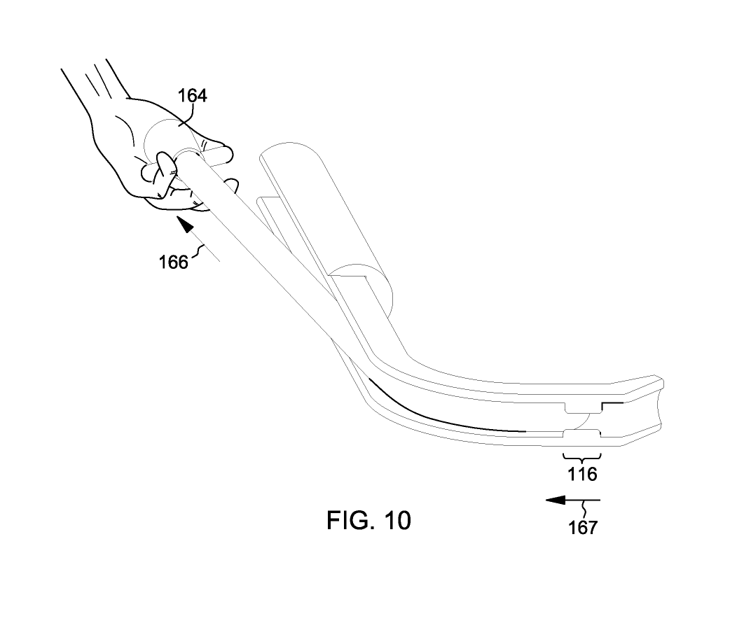

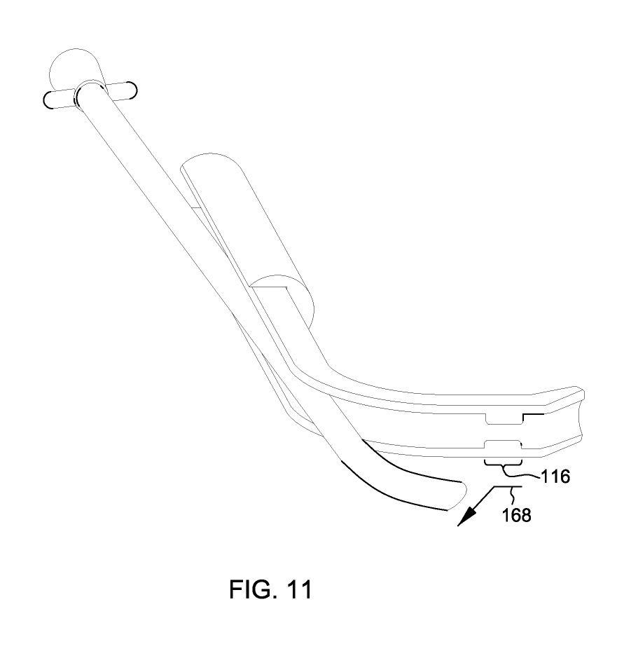

In another aspect of the invention, shown with reference to FIGS. 9-11, a method of detaching a flexible tube from a flexible tube holding device is provided. The method includes providing a system 150 for intubation. The system includes a flexible tube 107 and a flexible tube holding device 100. The flexible tube is preferably detachably held (e.g., see FIGS. 7 and 19) against a back side (106, FIG. 1) of a handle 102 of the flexible tube holding device. The holding device also includes a blade 108 coupled to the handle, the blade including a tube retention section 116 sized to accept at least most of a cross-section (120, FIGS. 2 and 4) of a tip portion (122, FIGS. 2 and 4) of the flexible tube and shaped to retain the tip portion (see FIGS. 2 and 4). The detaching method further includes releasing 165 the flexible tube from the back side of the flexible tube holding device, for example, by letting go if manually holding as shown in FIG. 9, or opening a quick-release closure mechanism (see FIG. 20), where present, and pulling 166 on a proximal end 164 of the flexible tube (see FIG. 10) to cause the flexible tube to slide (167, FIG. 10) proximally through and out (168, FIG. 11) of the tube retention section 116.

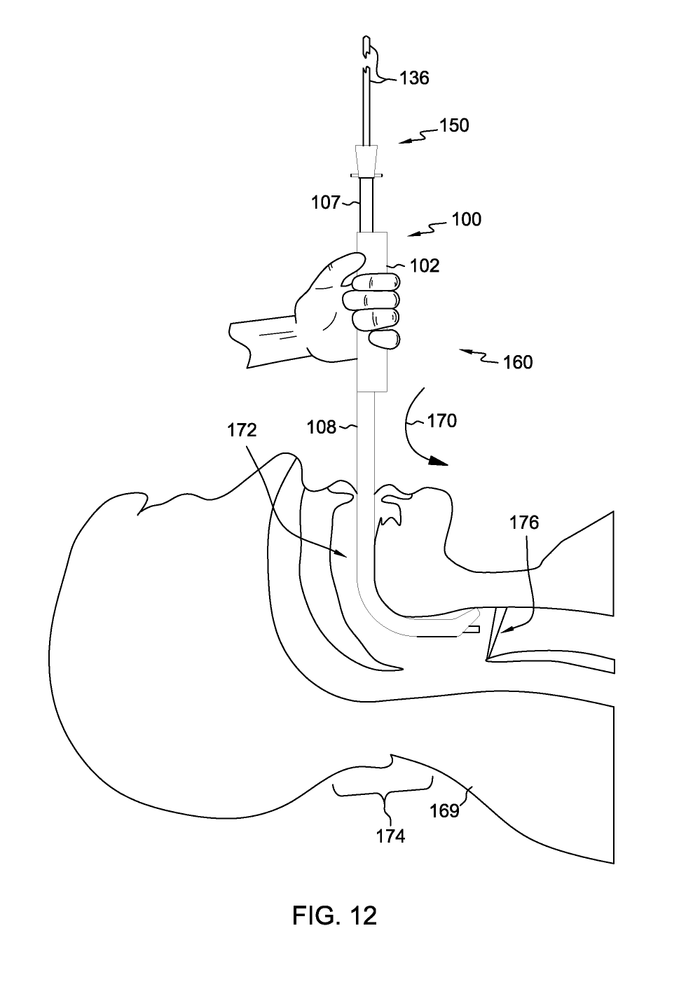

In still another aspect of the invention, a method of intubating a patient 169 is provided and described with reference to FIGS. 12-18. An intubation system according to the invention is provided, for example, system 150 in FIG. 12. In one example, the system includes a first flexible tube 107 appropriate for the patient and situation, a flexible tube holding device 100 and an intermediate intubation device 136 appropriate for the patient and situation. The flexible tube holding device includes, for example, a handle 102 and a blade 108 coupled to the handle. The blade includes a tube retention section (116, FIG. 1) sized to accept at least most of a cross-section (120, FIGS. 2 and 4) of a tip portion (122, FIGS. 2 and 4) of the first flexible tube, and shaped to retain the tip portion (see FIGS. 2 and 4). The first flexible tube is detachably held (e.g., manually 160) against a back side (106 and 114, FIG. 1) of the flexible tube holding device.

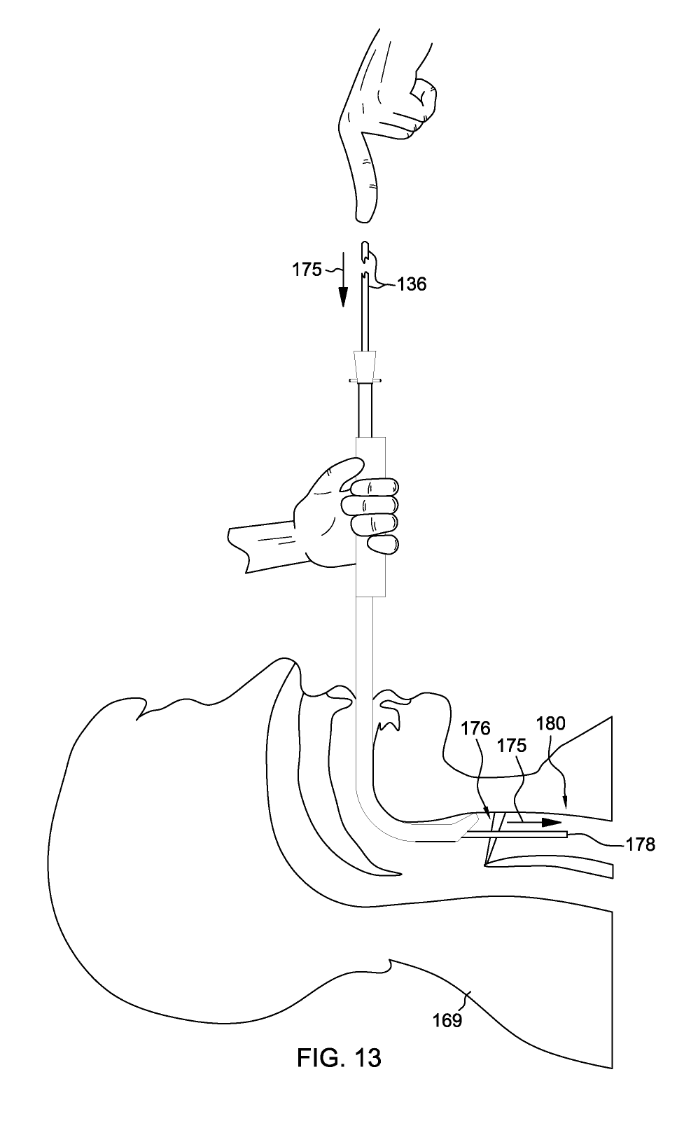

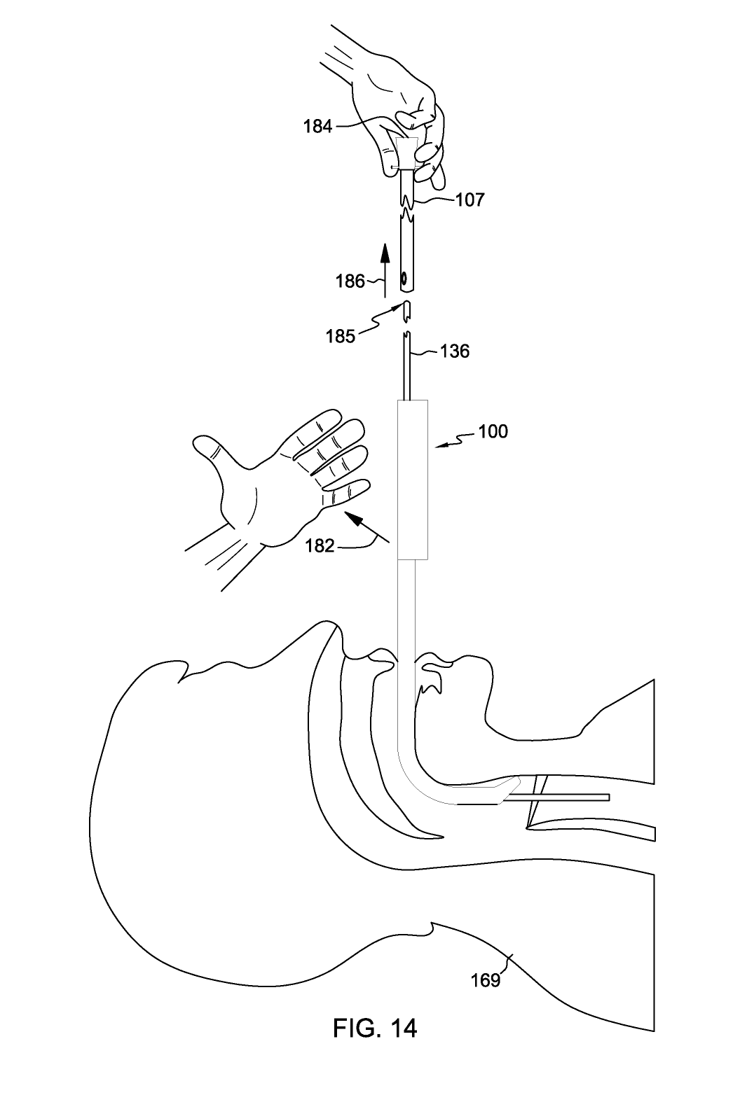

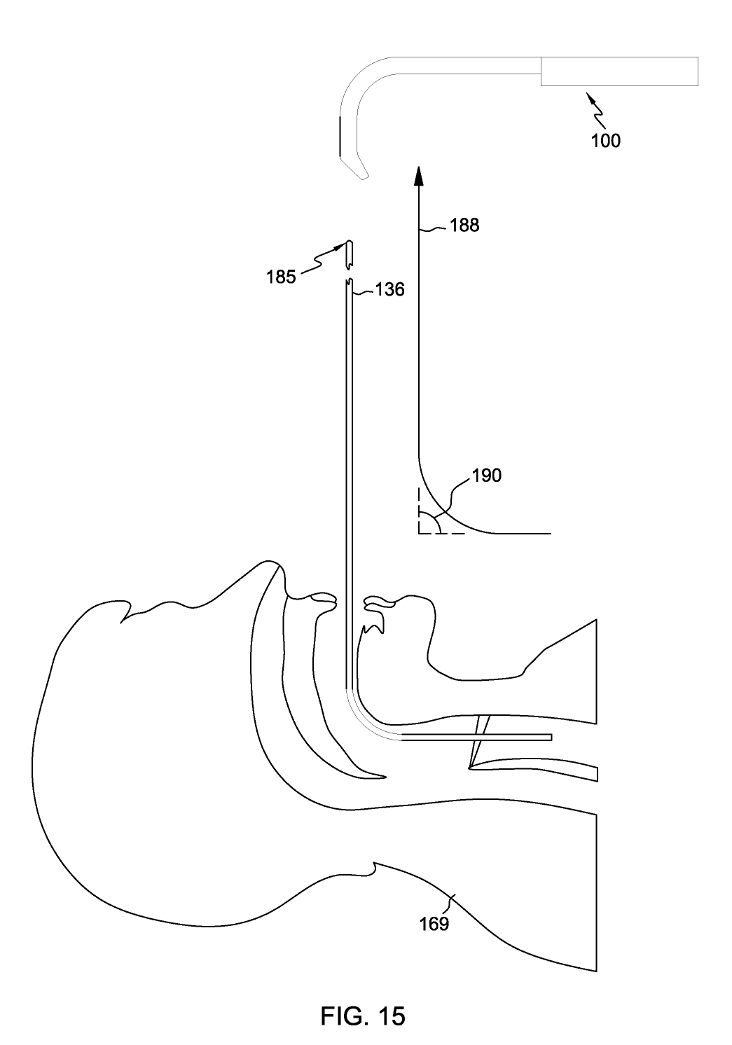

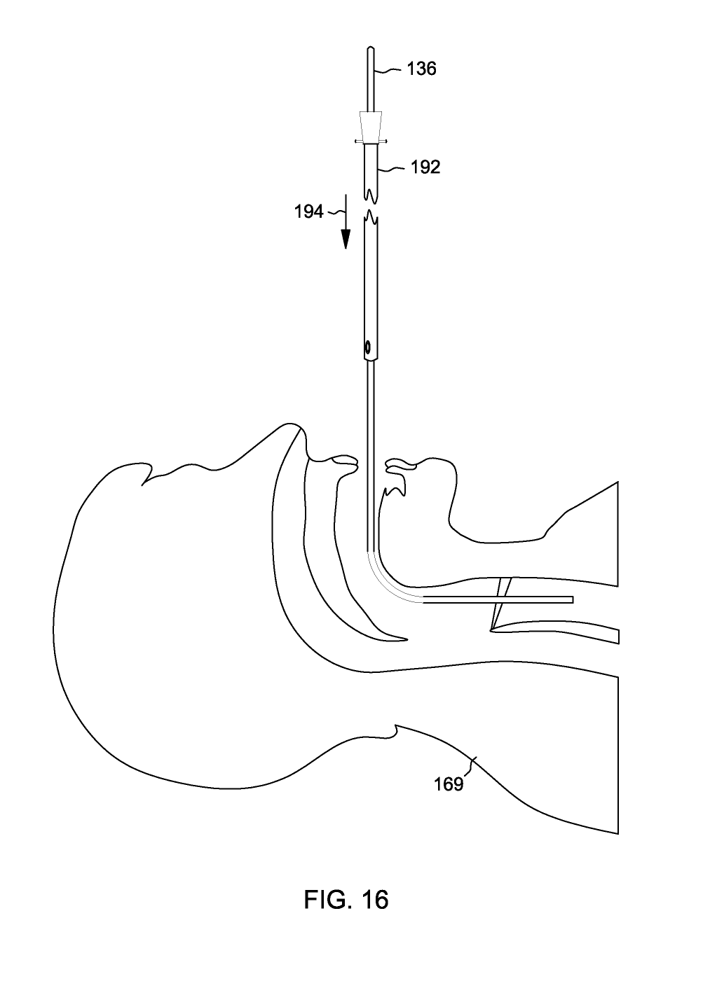

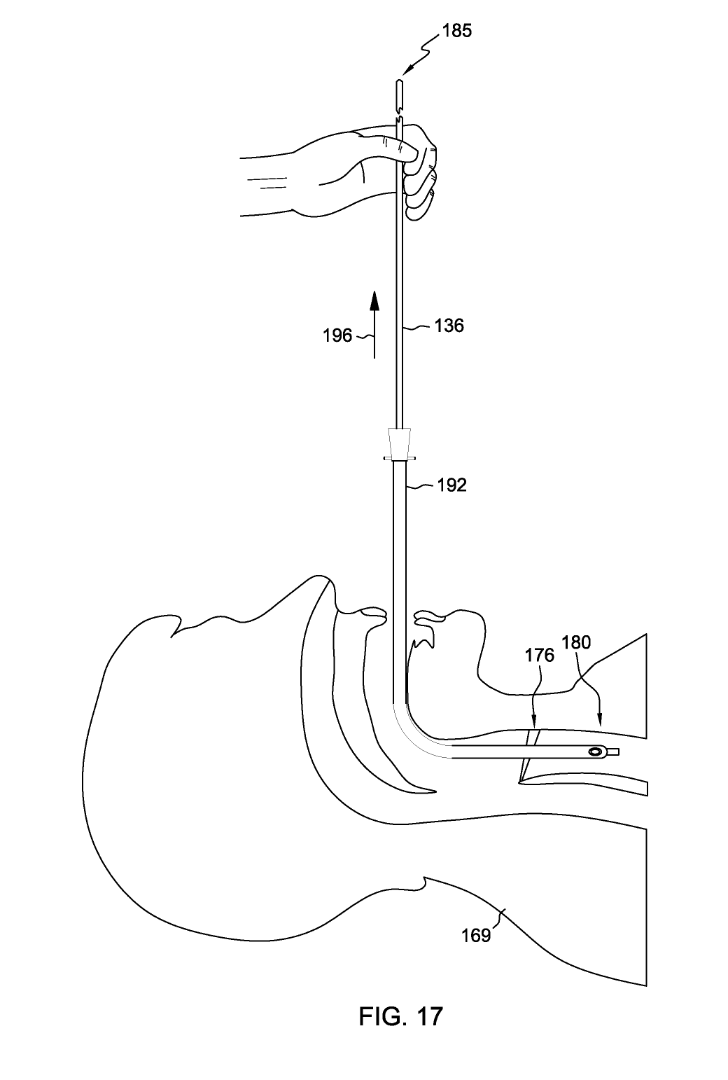

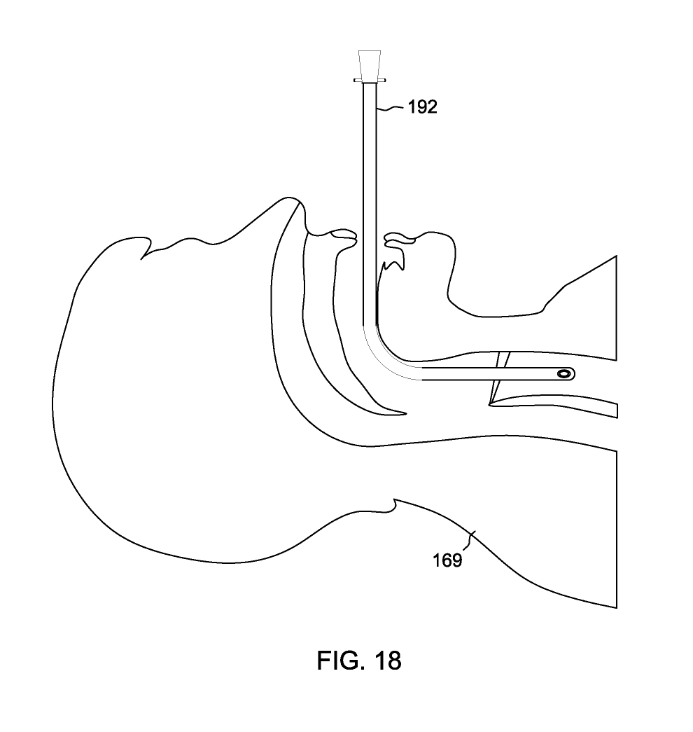

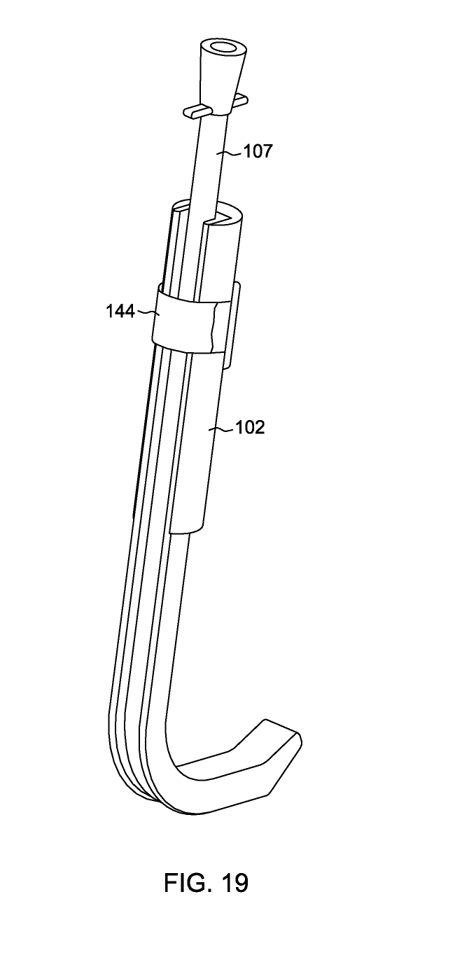

Continuing with the intubation method, the intubation system is inserted 170 into an upper airway 172 of the patient 169, the intubation system being maneuvered to expand the upper airway. In one example, conventional maneuvers for laryngoscopy may be used. Preferably, when using the version of the flexible tube holding device shown in the figures, the patient's head is in a neutral position 174 with respect to their neck. Returning to the intubation method, the patient's vocal cords 176 are visualized in some manner, discussed subsequently in more detail, prior to advancing (175, FIG. 13) a distal end 178 of the intermediate intubation device 136 between the vocal cords and into the patient's trachea 180 based on the visualization thereof. The first flexible tube is then released (182, FIG. 14) from the handle and blade of the flexible tube holding device. Further, in FIG. 14, a proximal end 164 of the first flexible tube is pulled on 186 to cause the first flexible tube to slide proximally out of the tube retention section, while minimizing movement of the intermediate intubation device, and removed as shown. In one example, as shown in FIG. 15, the flexible tube holding device 100 is removed by lifting and rotating 188 the same through an angle 190 of about 90 degrees, while leaving the intermediate intubation device 136 in place. As shown in FIG. 16, a second flexible tube 192 may then be slid 194 over the intermediate intubation device and, as shown in FIG. 17, through the vocal cords 176 and into the trachea 180 of the patient 169. The intermediate intubation device may then be removed by pulling 196 on a proximal end 198 thereof, leaving the second flexible tube in place as shown in FIG. 18.

It will be understood that the reason for the order of removal described with respect to FIGS. 14-18 was so that a different flexible tube than initially used could be employed for final intubation, for example, when the first flexible tube is not an endotracheal tube. If only one flexible tube will be used, then the order of removal is preferably the intermediate intubation device first, then the flexible tube holding device second, leaving the flexible tube in the patient's trachea. Alternatively, though not preferred as it is more cumbersome, the flexible tube holding device could be removed prior to the intermediate intubation device after disengaging the flexible tube and intermediate intubation device from the flexible tube holding device. Preferably, the intermediate intubation device is removed prior to the flexible tube holding device, as it is less cumbersome, but they can be removed in opposite order as well.

In one example, the flexible tube of the method of FIGS. 12-18 is held in place manually, and releasing the flexible tube is accomplished by letting go of the handle of the holding device (e.g., as shown in FIG. 9). In another example, the flexible tube holding device further includes a quick-release closure mechanism (e.g., mechanism 144 as shown in FIGS. 19 and 20) coupled to the handle to hold the flexible tube at a back side (106, FIG. 1) of the handle. Where the quick-release mechanism is present, releasing the flexible tube is accomplished by opening (145, FIG. 20) the quick-release closure.

In another example, the handle includes a groove (104, FIG. 2) along a length of a back side (106, FIG. 1) thereof, the groove sized to at least partially accept a desired flexible tube (e.g., flexible tube 107). Further, in this example, the blade 108 includes a proximal blade section (110, FIG. 1) coupled to the handle and having an opening (112,

FIG. 2) along a back side (114, FIG. 1). The blade also includes a tube retention section (116, FIG. 1) coupled to the proximal blade section at an end (118, FIG. 1) thereof opposite the handle and along the opening. The tube retention section is sized to accept at least most of a cross-section (120, FIGS. 2 and 4) of a tip portion (122, FIGS. 2 and 4) of the flexible tube and shaped (see FIG. 4) to retain the tip portion. In the present example, the flexible tube is also manually detachably held (160, FIG. 7) in the groove, and the releasing includes releasing the flexible tube from the groove.

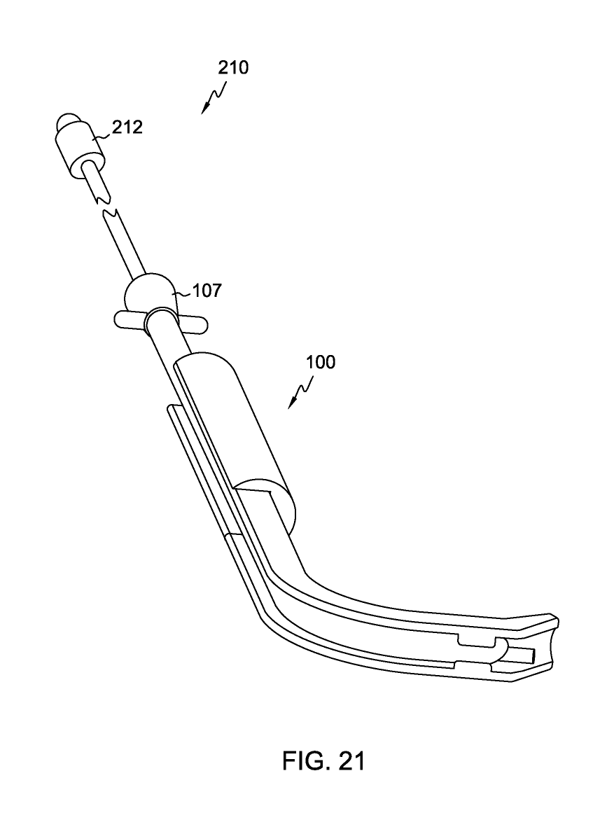

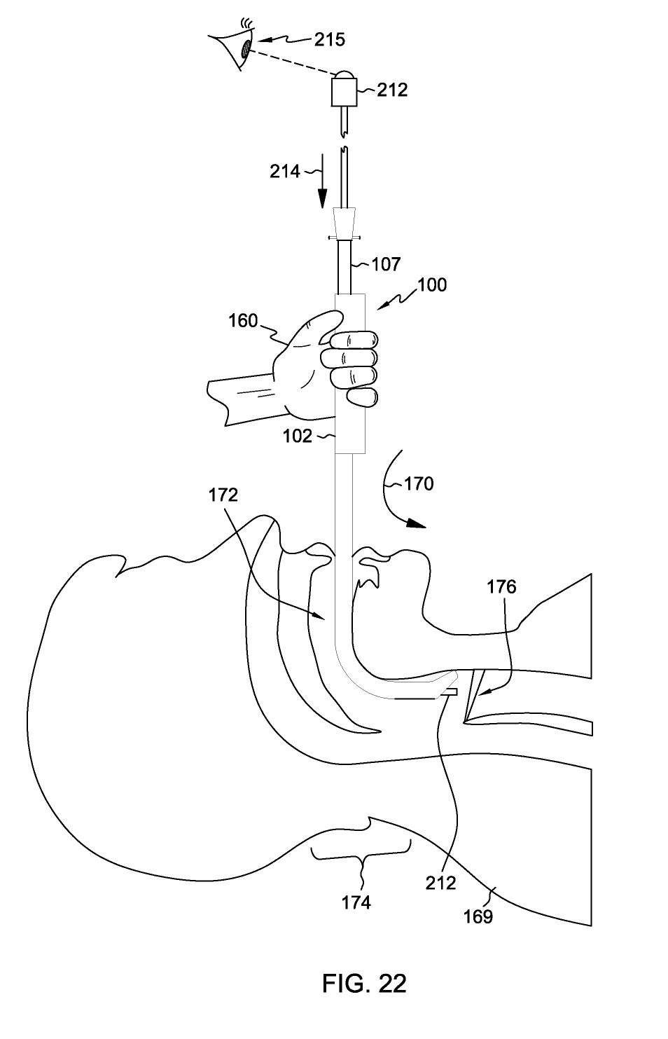

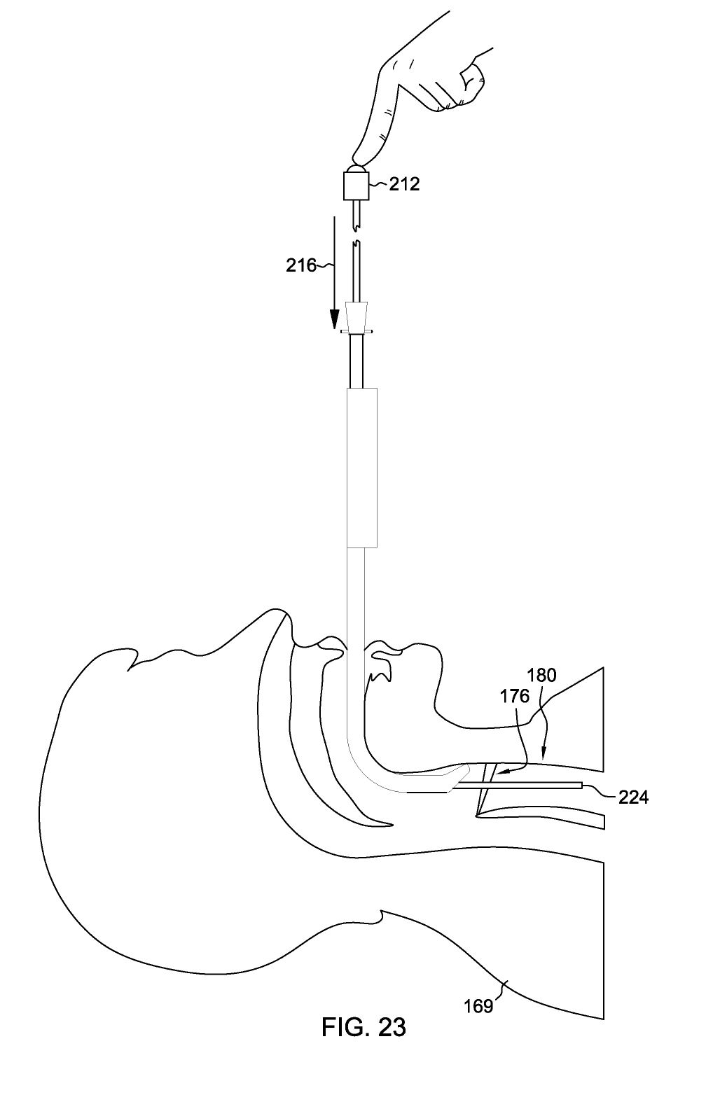

In another example, the method of intubating may include, as shown in FIG. 21, an intubation system 210 similar to that of FIG. 2, except the intermediate intubation device takes the form of an elongated imaging device 212, and including a flexible tube 107 and a flexible tube holding device 100. The intubation method, described with respect to FIGS. 22-29, further includes positioning (214, FIG. 22) the elongated imaging device to image (e.g., with eye 215) the vocal cords 176 of the patient 169 prior to advancing (216, FIG. 23) the elongated imaging device through the vocal cords and into the trachea 180 of the patient.

In the example of FIGS. 22-29, the first flexible tube 107 and the second flexible tube are the same flexible tube, i.e., there is only one flexible tube, and the elongated imaging device 212 is sized to fit within the flexible tube. In this example, the tube retention section (116, FIG. 1) may include a retention gap (130, FIG. 4) of less than an outer diameter (120, FIG. 4) of the flexible tube and greater than an external diameter (134, FIG. 4) of the intermediate intubation device.

As best shown in FIG. 22, the flexible tube holding device includes, for example, a handle 102 and a blade 108 coupled to the handle. The blade includes a tube retention section (116, FIG. 1) sized to accept at least most of a cross-section (120, FIGS. 2 and 4) of a tip portion (122, FIGS. 2 and 4) of flexible tube 107, and shaped to retain the tip portion (see FIGS. 2 and 4). The flexible tube is detachably held (e.g., manually 160) against a back side (106 and 114, FIG. 1) of the flexible tube holding device.

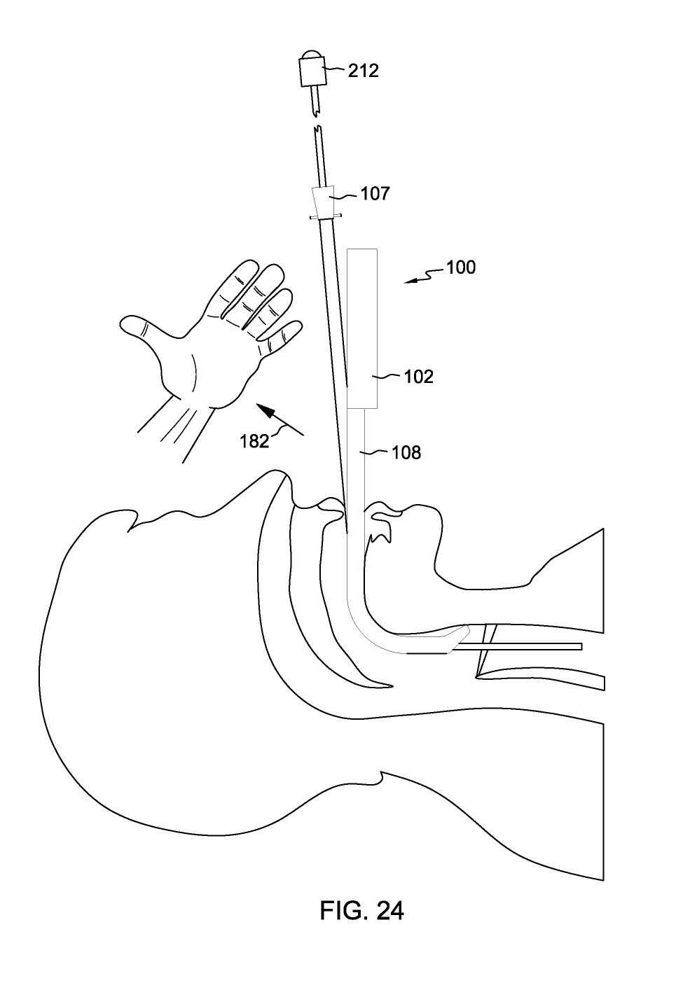

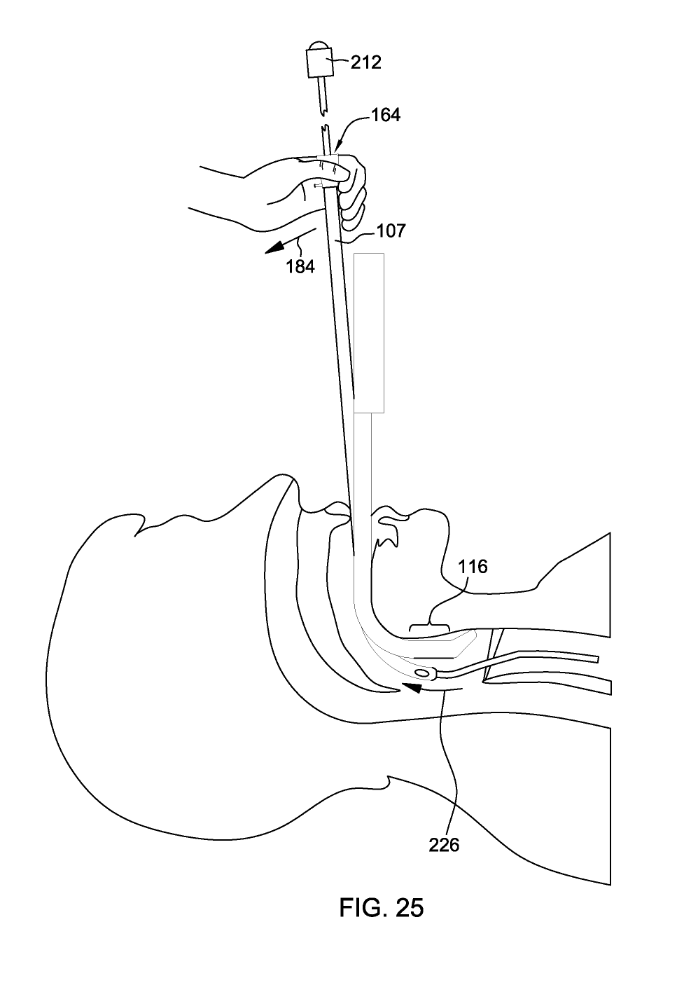

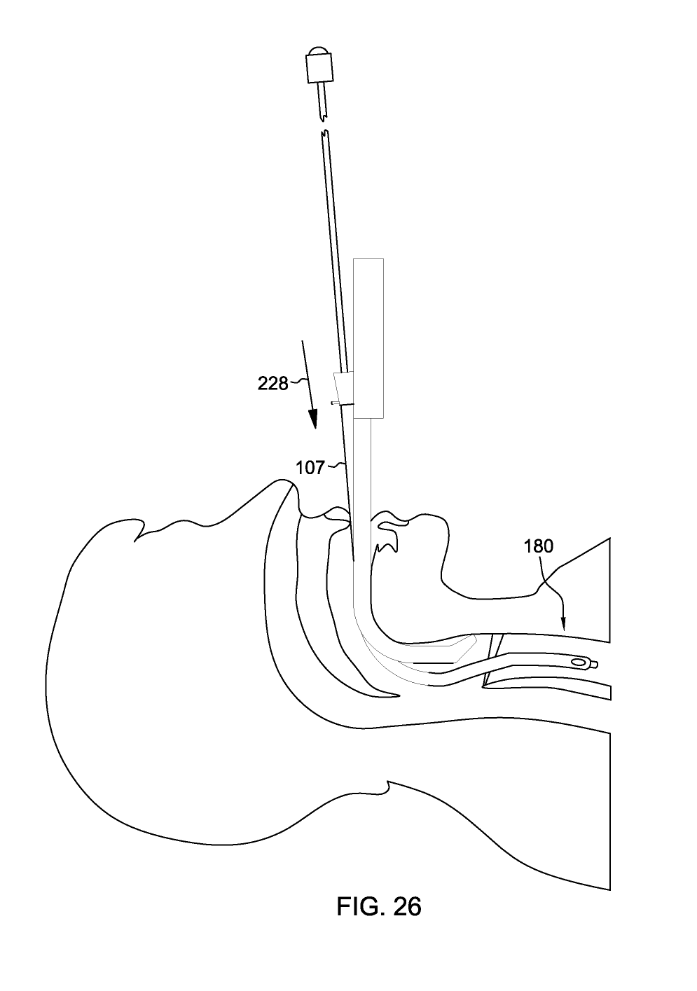

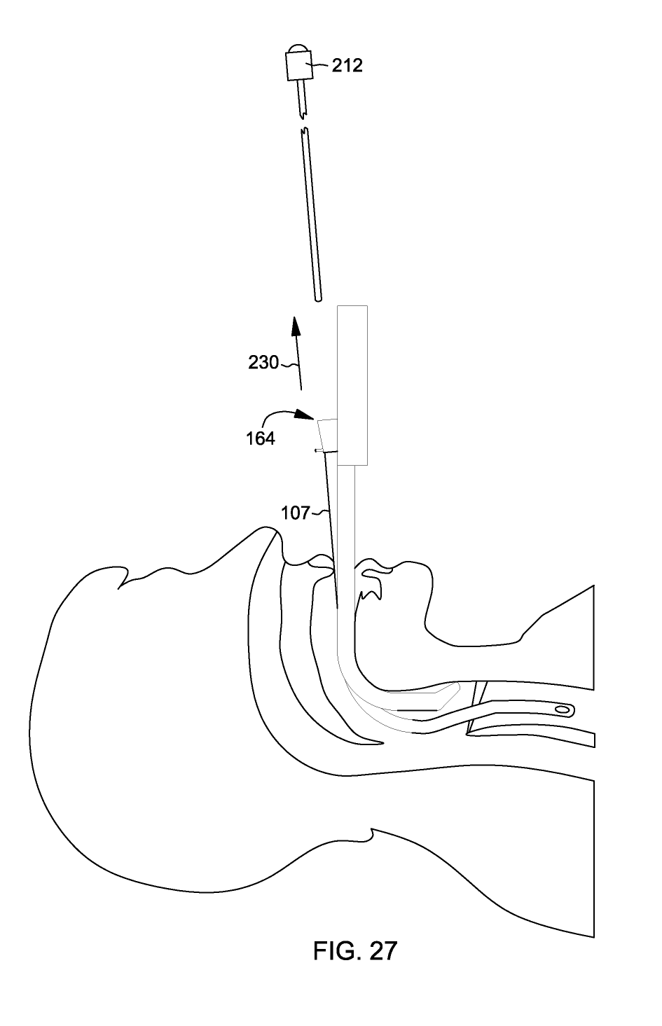

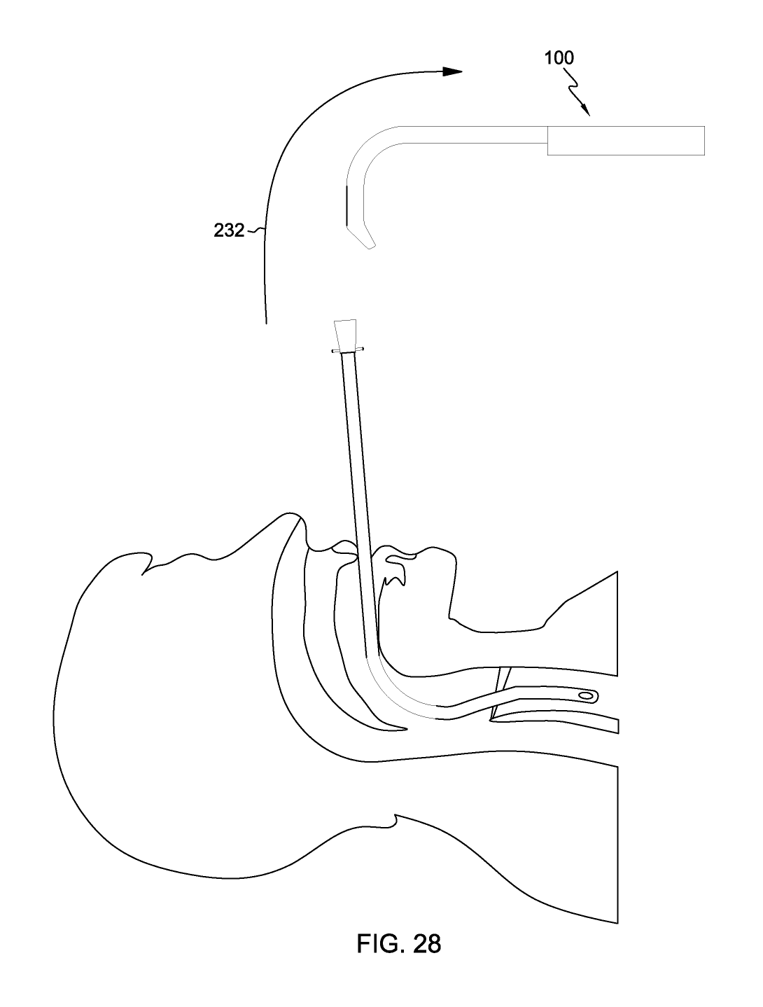

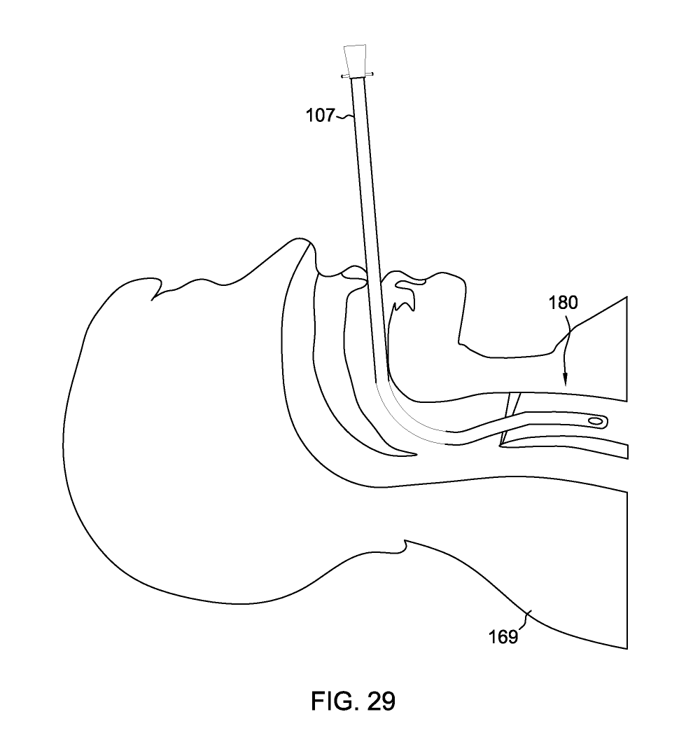

Continuing with the intubation method of FIGS. 22-29, the intubation system 210 is inserted (170, FIG. 22) into an upper airway 172 of the patient 169, the intubation system being maneuvered to expand the upper airway. In one example, conventional maneuvers for laryngoscopy may be used. Preferably, when using the version of the flexible tube holding device shown in the figures, the patient's head is in a neutral position (174, FIG. 22) with respect to their neck. Returning to the intubation method, the patient's vocal cords (176, FIG. 22) are visualized in some manner, discussed elsewhere in more detail, prior to advancing (216, FIG. 23) a distal end (224, FIG. 23) of the intermediate intubation device between the vocal cords and into the patient's trachea 180 based on the visualization thereof. The flexible tube is then released 182, as shown in FIG. 24, from the handle and blade of the flexible tube holding device. A proximal end 164 of the flexible tube is pulled on 184, to cause the flexible tube to slide (226, FIG. 25) proximally out of the tube retention section 116 while minimizing movement of the elongated imaging device 212. The pulling causes the tip of the flexible tube to move out of the tube retention section, the released flexural tension causing the tip of the flexible tube to move out of the opening of the proximal blade section (112, FIG. 2) and away from the flexible tube holder. The operator, by moving the flexible tube holding device, and/or the flexible tube, may also contribute to the movement of the flexible tube out of the groove. No longer held inside the tube retention section by the tip of the flexible tube (122, FIG. 2), the body of the intermediate intubation device moves through the retention gap (130, FIG. 4) and out of the tube retention section. The flexible tube is then slid (228, FIG. 26) over the elongated imaging device and into the trachea 180. The elongated imaging device is removed by sliding (230, FIG. 27) it out of the proximal end 164 of the flexible tube. The flexible tube holding device is removed by lifting the flexible tube holding device 100 while rotating it through an angle of about 90 degrees (232, FIG. 28), similar to the removal described with respect to FIG. 15. The flexible tube is left in the trachea of the patient, as shown in FIG. 29.

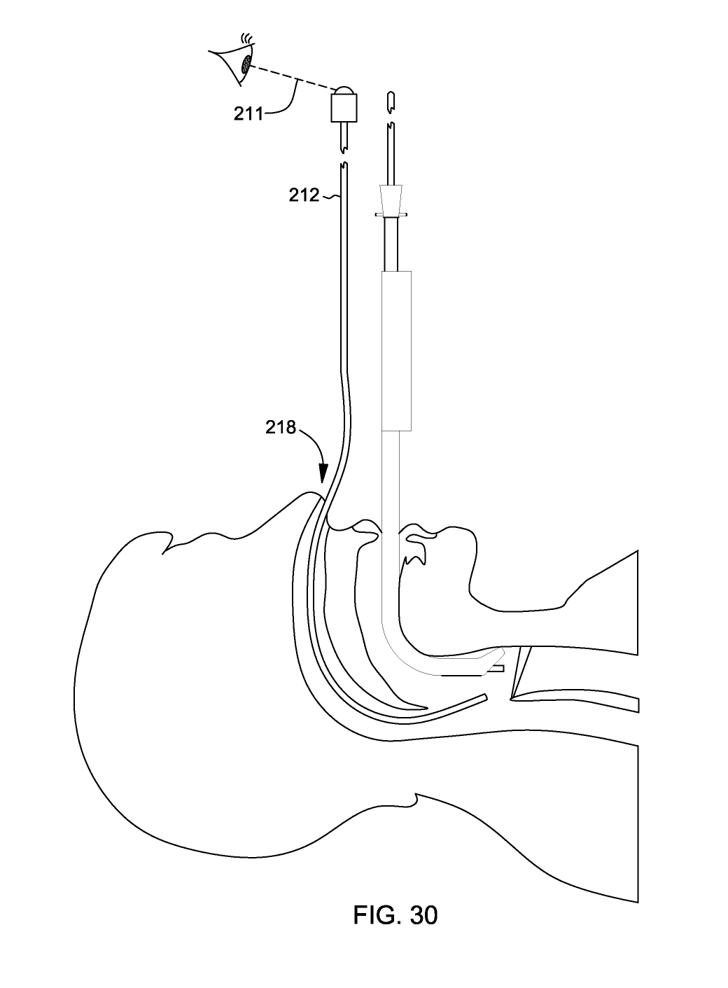

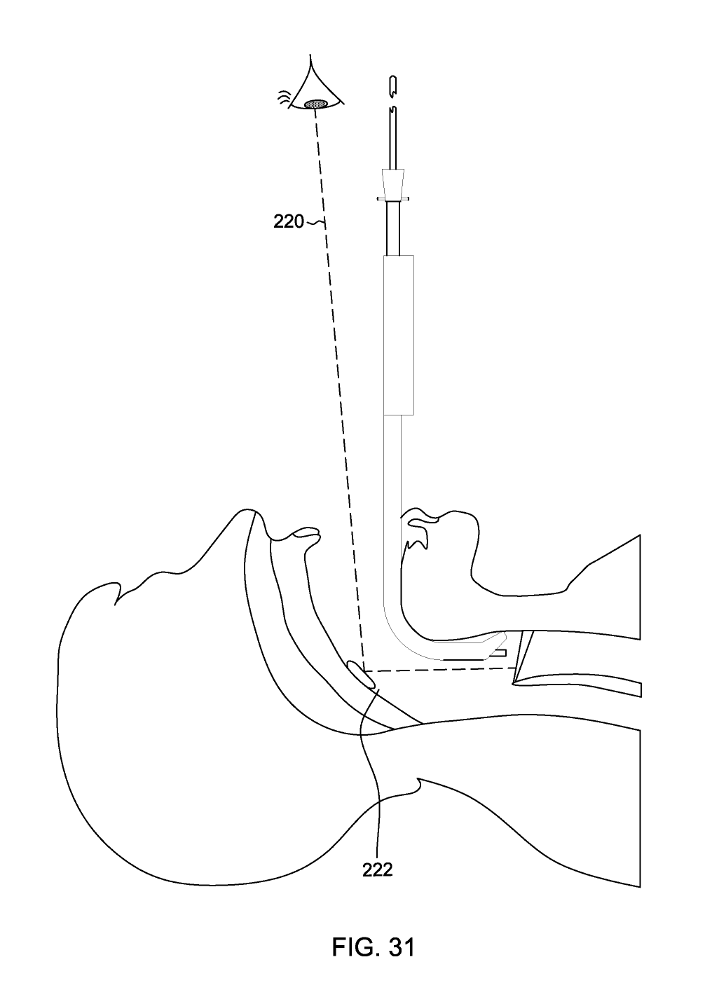

Visualizing the vocal cords may include, for example, one of visualization (211, FIG. 30) with an elongated imaging device (212, FIG. 22) situated within the first flexible tube (107, FIG. 22), an elongated imaging device (212, FIG. 30), instead of being situated within the first flexible tube, being inserted through the nasal cavity (218, FIG. 30), indirect visualization by reflection (220, FIG. 31) using a reflective surface 222 placed in the back of the throat, the use of a rigid videoscope, the use of a standard laryngoscope, the use of a fiber optic bronchoscope situated outside the first flexible tube, and the use of a flexible, elongated videoscopic device situated outside the first flexible tube.

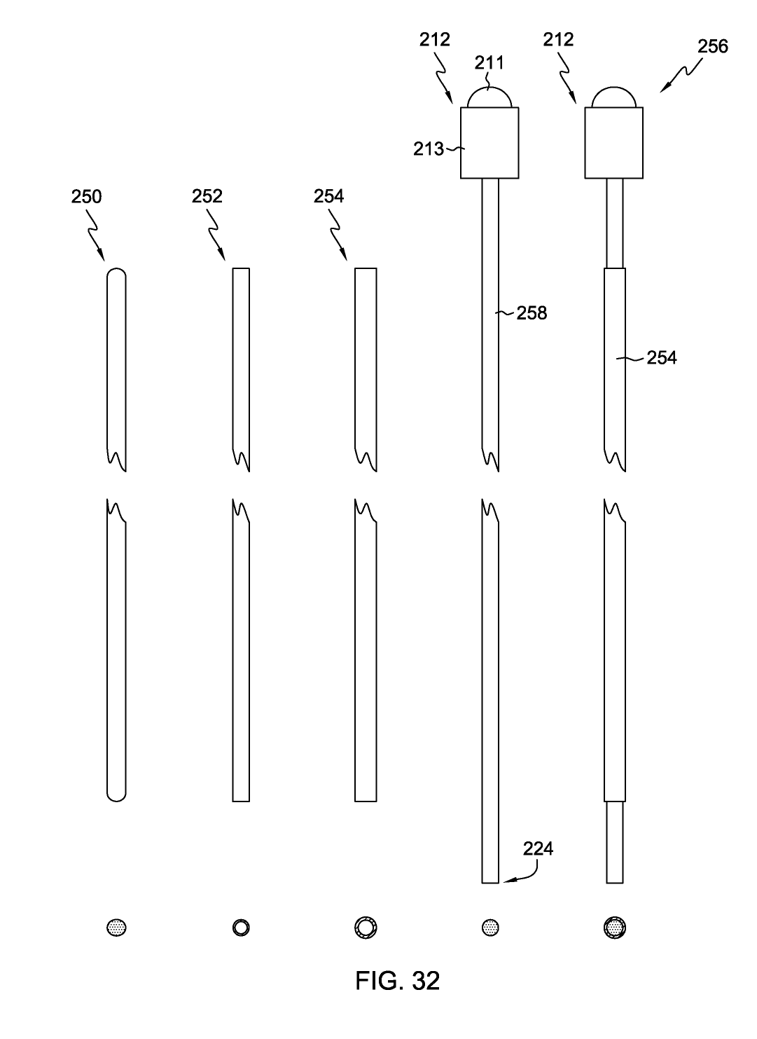

In another example, as shown in FIG. 32, the intermediate intubation device 136 includes one of an intubating bougie 250, an exchange catheter 252, a hollow intubation catheter 254 sized to accept an elongated imaging device 212, in this example a fiberoptic bronchoscope, and an elongated imaging device within a hollow intubation catheter 256. The imaging device includes: a proximal lens 211 for viewing; a handle 213 housing, for example, a battery power source and a light source (not shown); a flexible shaft 258 containing fibers for transmitting light distally and images proximally; control cables (not shown); and a tip 224 equipped to acquire optical images and to provide illumination. The handle may also include levers (not shown) for pulling on the control cables to alter the orientation of the tip. Where the intermediate intubation device includes an elongated imaging device, the elongated imaging device may take the form of, for example, a fiber optic bronchoscope or a flexible, elongated videoscope.

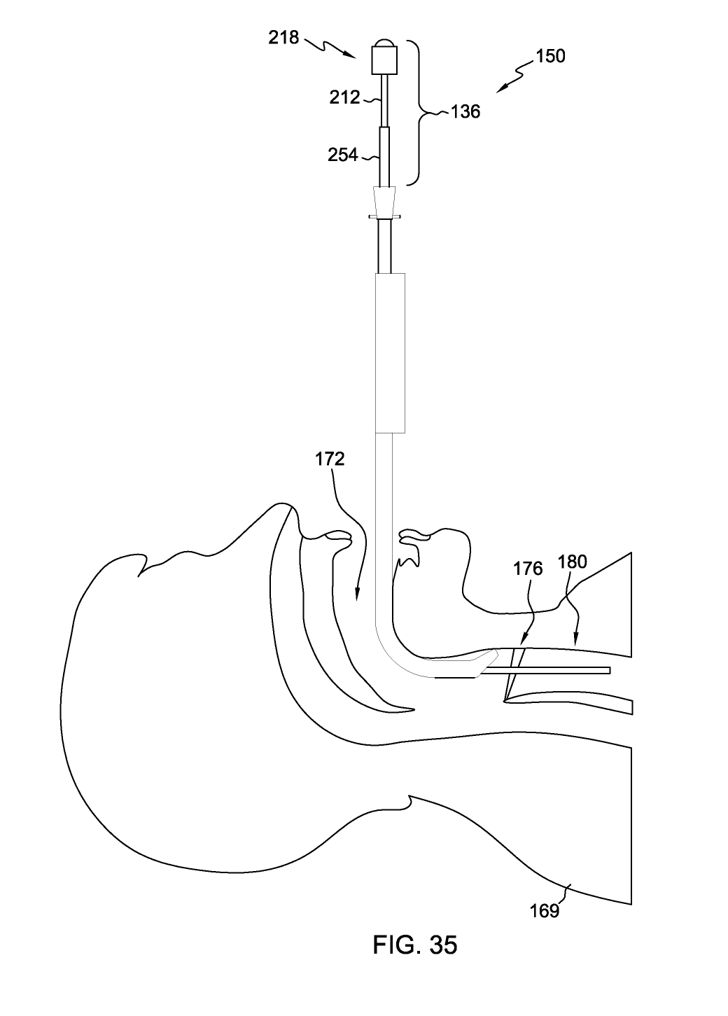

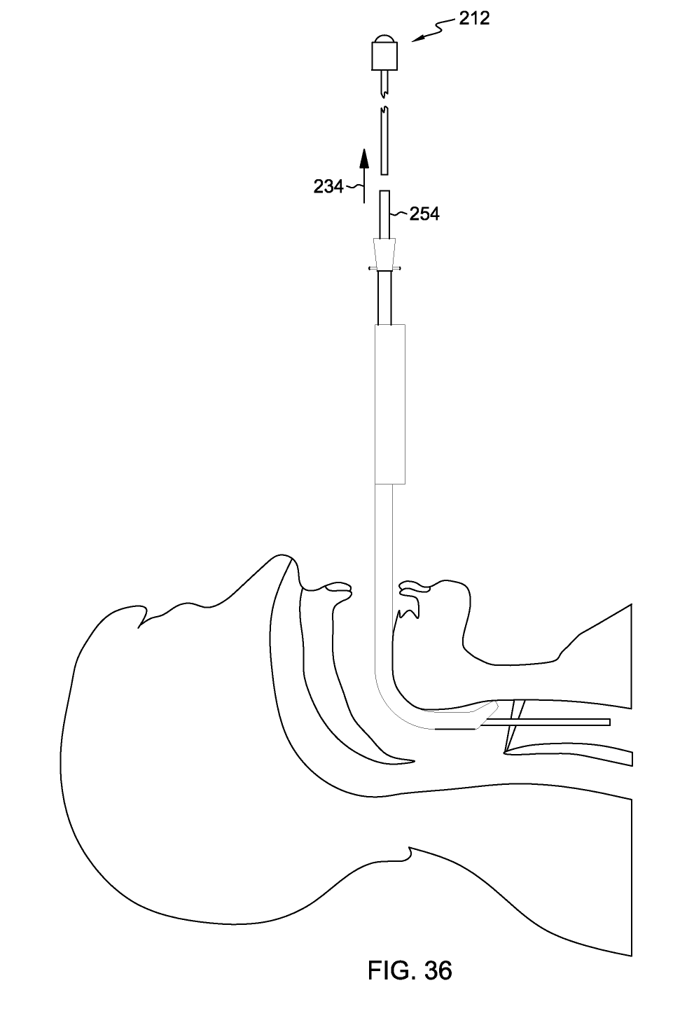

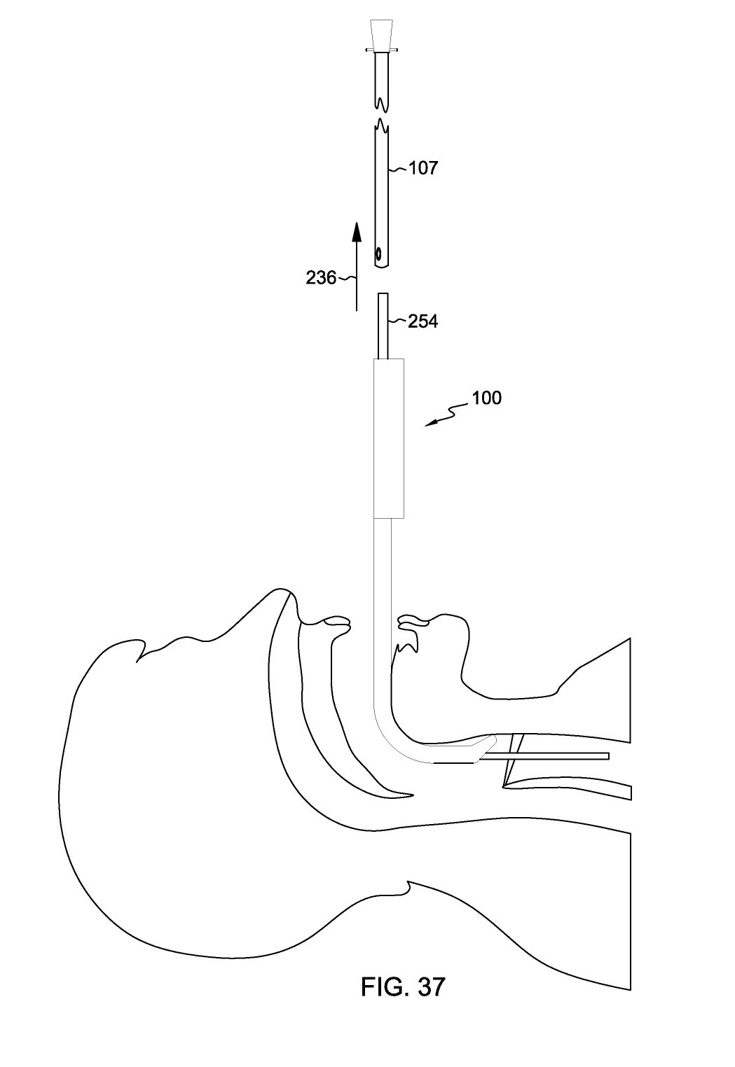

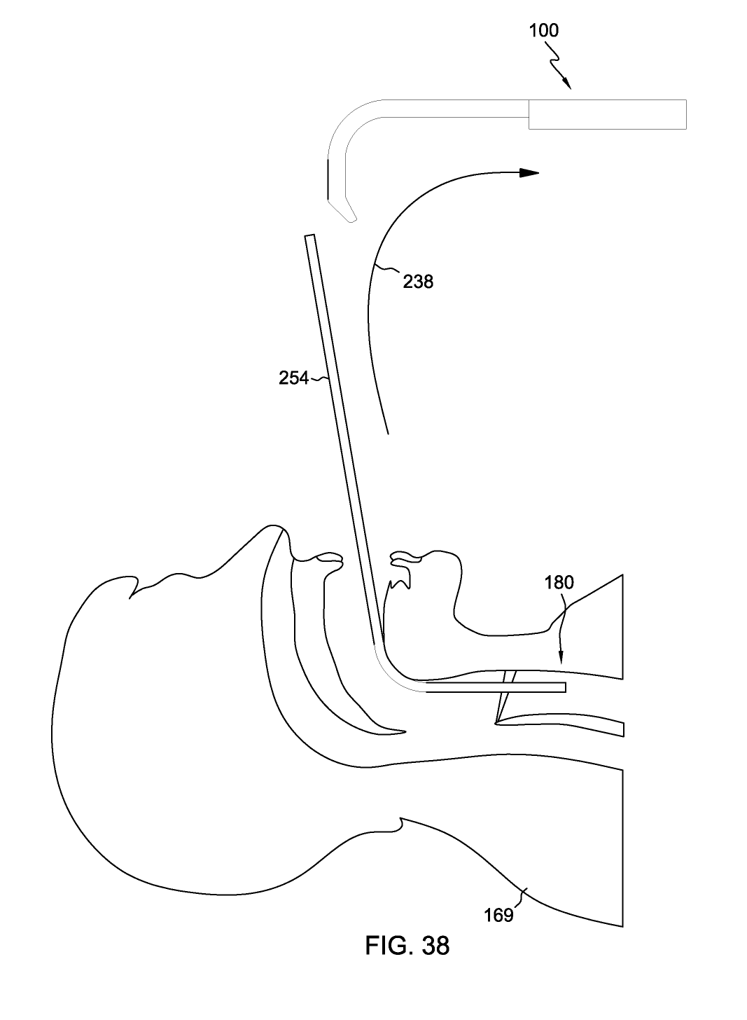

In yet another example, shown in FIGS. 35-38, where the intermediate intubation device 136 includes an elongated imaging device 212 within a hollow intubation catheter 254, the method may further include, after removing 234 the elongated imaging device (FIG. 36), removing (236, FIG. 37) the first flexible tube 107 and removing (238, FIG. 38) the flexible tube holding device 100, leaving the hollow intubation catheter in the trachea 180 of the patient 169.

It is intended that different versions of the flexible tube holding device may be produced to accommodate different sizes of endotracheal tubes, so that dimensions of the various parts will differ from one version to another. It is intended further that different versions of the flexible tube holding device may be produced to accommodate different kinds of airway anatomy, or to accommodate the different preferences of various operators, so that the dimensions and shape of the blade, and of the subunits making up the blade, will vary from version to version of the blade.

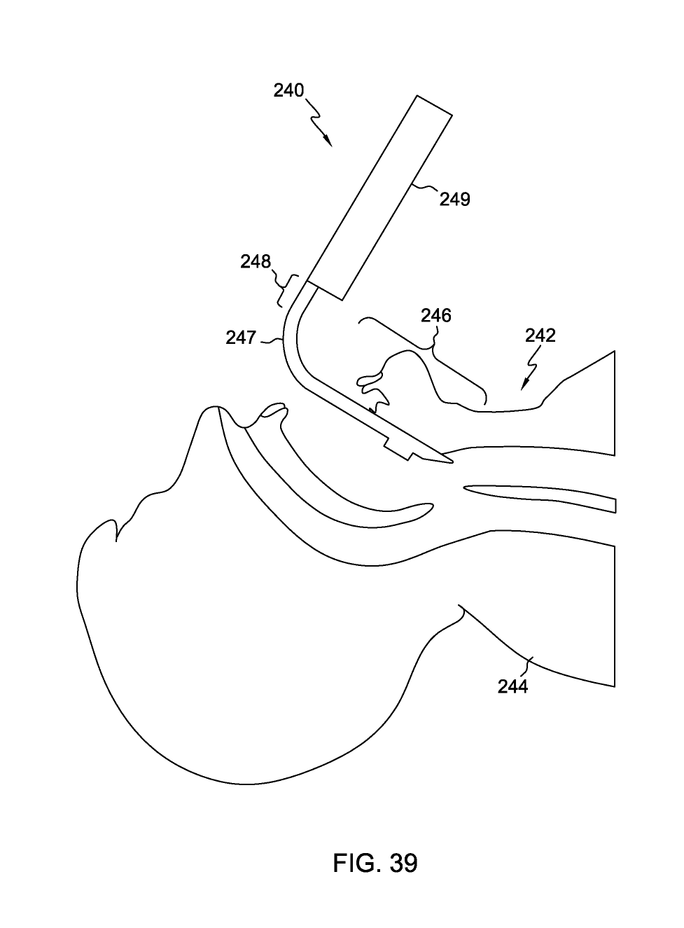

With the above in mind, another example of the flexible tube holding device 240 is shown in FIG. 39 after placement in an upper airway 242 of a patient 244 to be intubated. Of note, in this example, the insertion section 246—the portion of the blade 247 inserted into the upper airway—is relatively straight and the section 248 of the blade coupled to the handle 249 is shorter than that shown in FIG. 1, but the design is otherwise similar to that shown in FIGS. 1 and 2.

FIGS. 35-38 depict steps in the intubation method of FIGS. 12-18 in which the intermediate intubation device 136 includes an elongated imaging device, similar to the example of FIG. 22, but including a fiber optic bronchoscope 218 inside a hollow intubation catheter 254. FIG. 35 shows the intubation system 150 in the upper airway 172 of the patient 169, after the intermediate intubation device has been advanced through the vocal cords 176 and into the trachea 180. In FIG. 36, the fiber optic bronchoscope is being removed 234 from the catheter. In FIG. 37, the flexible tube 107 is being removed, leaving catheter 254, and flexible tube holding device 100. In FIG. 38, the flexible tube holding device is being removed. The initial flexible tube 107 or a different flexible tube may then be passed over the catheter 254, and into the trachea, in the manner shown in FIGS. 16 and 17.

Different versions of the flexible tube holding device can be produced with various materials and in various combinations of materials. For example, the flexible tube holing device may be manufactured as a single piece (preferred), using a medical-grade plastic, either by direct three-dimensional printing or by injection molding, for example. Alternatively, the flexible tube holding device could be produced in a combination of materials, for example, the blade comprised of stainless steel, the handle of plastic, with the two parts connected in some reliable manner, e.g., by a flange-slot mechanism.

It should be noted that other flexible medical tubes besides an endotracheal tube could be used with the present flexible tube holding device. Similarly, although the flexible tube holding device typically would be used with a fiber optic bronchoscope, other flexible airway devices, for example, an endotracheal exchange catheter or an Aintree intubation catheter, could also be used.

While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

1. A flexible tube holding device, comprising:
a handle, wherein the handle comprises a groove along a length of a back side thereof, the groove sized to at least partially accept a desired flexible tube; and
a blade coupled to the handle, the blade comprising:
a proximal blade section coupled to the handle and having an opening along a back side thereof; and
a tube retention section coupled to the proximal blade section at an end thereof opposite the handle and along the opening, wherein the tube retention section is sized to accept at least most of a cross-section of a tip portion of the flexible tube and shaped to retain the tip portion.

2. The device of claim 1, further comprising a tip section coupled to the tube retention section at an end thereof opposite the proximal blade section.

3. The device of claim 2, wherein the tip section comprises an upward tilt.

4. The device of claim 1, wherein the blade comprises a generally J shape.

5. The device of claim 4, wherein the blade curves to about a 90° angle with respect to the handle.

6. The device of claim 1, wherein the proximal blade section comprises a generally U or C shape in cross-section with an opening along a back side thereof.

7. The device of claim 1, wherein the proximal blade section comprises a plurality of subsections of different sizes and/or cross-sections.

8. The device of claim 1, wherein the proximal blade section is curved.

9. The device of claim 1, wherein the tube retention section comprises a retention gap of less than an outside diameter of the flexible tube and greater than an outside diameter of a desired intermediate intubation device.

10. The device of claim 9, wherein the retention gap is formed by at least two flanges extending inward from opposite sides of the blade.

11. The device of claim 10, wherein the at least two flanges are substantially rigid.

12. The device of claim 9, wherein the retention gap is sized approximately equal to an internal diameter of the flexible tube.

13. The device of claim 1, wherein the flexible tube holding device further comprises a quick-release closure mechanism coupled to the handle to hold a flexible tube in the groove, and wherein the flexible tube is held to the back side of the handle by the quick-release closure mechanism.

14. The device of claim 1, wherein the proximal blade section comprises a generally flat cross-section.

15. The device of claim 1, wherein the tube retention section is sized to hold no more than about 4 centimeters of a length of the tip portion of the flexible tube.

16. An intubation system, comprising:
a flexible tube;
an intermediate intubation device sized to fit within the flexible tube; and
a flexible tube holding device, comprising:
a handle, wherein the handle comprises a groove along a length of a back side thereof, the groove sized to at least partially accept the flexible tube; and
a blade coupled to the handle, the blade comprising:
a proximal blade section coupled to the handle and having an opening along a back side thereof; and
a tube retention section coupled to the proximal blade section at an end thereof opposite the handle and along the opening, wherein the tube retention section is sized to accept at least most of a cross-section of a tip portion of the flexible tube and shaped to retain the tip portion.

17. The system of claim 16, further comprising a tip section coupled to the tube retention section at an end thereof opposite the proximal blade section.

18. The system of claim 17, wherein the tip section comprises an upward tilt.

19. The system of claim 16, wherein the blade comprises a generally J shape.

20. The system of claim 19, wherein the blade curves to about a 90° angle with respect to the handle.

21. The system of claim 16, wherein a cross-section of the proximal blade section comprises a generally U or C shape.

22. The system of claim 16, wherein the proximal blade section comprises a plurality of subsections of different sizes and/or cross-sections.

23. The system of claim 16, wherein the proximal blade section is curved.

24. The system of claim 16, wherein the tube retention section comprises a retention gap of less than an outside diameter of the flexible tube and greater than an outside diameter of the intermediate intubation device.

25. The system of claim 24, wherein the retention gap is formed by at least two flanges extending inward from opposite sides of the blade.

26. The system of claim 25, wherein the at least two flanges are substantially rigid.

27. The system of claim 16, wherein the retention gap is sized approximately equal to an internal diameter of the flexible tube.

28. The system of claim 16, wherein the flexible tube holding device further comprises a quick-release closure mechanism coupled to the handle to hold a flexible tube in the groove, and wherein the flexible tube is held to the back side of the handle by the quick-release closure mechanism.

29. The system of claim 16, wherein the proximal blade section comprises a generally flat cross-section.

30. The system of claim 16, wherein the tube retention section is sized to hold no more than about 4 centimeters of a length of the tip portion of the flexible tube.

31. A method of assembling an intubation system, the method comprising:
providing a flexible tube;
providing a flexible tube holding device, the device comprising:
a handle, wherein the handle comprises a groove along a length of a back side thereof, the groove sized to at least partially accept the flexible tube; and
blade coupled to the handle, the blade comprising:
a proximal blade section coupled to the handle and having an opening along a back side thereof; and
a tube retention section coupled to the proximal blade section at an end thereof opposite the handle and along the opening, wherein the tube retention section is sized to accept at least most of a cross-section of a tip portion of the flexible tube and shaped to retain the tip portion;
inserting the tip portion of the flexible tube into the tube retention section of the device;
bending the flexible tube into the groove of the handle;
causing the flexible tube to be detachably held in the groove and the opening; and
inserting an intermediate intubation device into a proximal end of the flexible tube and advancing it at least partly into the flexible tube.

32. The method of claim 31, wherein the proximal blade section is curved.

33. The method of claim 31, wherein the tube retention section comprises a retention gap of less than an external diameter of the flexible tube and greater than an external diameter of a desired intermediate intubation device.

34. The method of claim 31, wherein causing the flexible tube to be detachably held comprises holding the flexible tube in the groove of the handle manually.

35. The method of claim 31, wherein the device further comprises a quick release closure mechanism coupled to the handle to hold the flexible tube in the groove at the back side of the handle, and wherein causing the flexible tube to be detachably held comprises closing the quick-release closure.

36. The method of claim 31, wherein the tube retention section is sized to accept no more than about 4 centimeters of a length of the tip portion of the flexible tube.

37. A method of detaching a flexible tube from a flexible tube holding device, the method comprising:
providing a system for intubation, the system comprising:
a flexible tube; and
a flexible tube holding device, wherein the flexible tube is detachably held against a back side of the flexible tube holding device, the flexible tube holding device comprising:
a handle; and
a blade coupled to the handle, the blade comprising a tube retention section sized to accept at least most of a cross-section of a tip portion of the flexible tube and shaped to retain the tip portion;
releasing the flexible tube from the back side of the flexible tube holding device; and
pulling on a proximal end of the flexible tube to cause the flexible tube to slide proximally out of the tube retention section.

38. The method of claim 37, wherein the device further comprises a quick-release closure mechanism coupled to the handle to detachably hold the flexible tube at the back side of the handle, and wherein releasing the flexible tube comprises opening the quick-release closure.

39. The method of claim 37, wherein the handle comprises a groove along a length of a back side thereof, the groove sized to at least partially accept a desired flexible tube, wherein the blade comprises a proximal blade section coupled to the handle and having an opening along a back side thereof, wherein the blade further comprises a tube retention section coupled to the proximal blade section at an end thereof opposite the handle and along the opening, wherein the tube retention section is sized to accept at least most of a cross-section of a tip portion of the flexible tube and shaped to retain the tip portion, wherein the flexible tube is also detachably held in the groove, and wherein the releasing comprises releasing the flexible tube from the groove.

40. The method of claim 39, wherein the intubation system further comprises an intermediate intubation device situated at least partly in the flexible tube, and wherein the pulling causes the flexible tube to slide proximally over a shaft of the intermediate intubation device and out of the tube retention section.

41. The method of claim 40, wherein prior to the pulling the intermediate intubation device is situated in a trachea of a patient, and wherein the pulling comprises leaving the distal end of the intermediate intubation device in the trachea, the method further comprising removing the intermediate intubation device from the flexible tube.

42. The method of claim 41, wherein the intermediate intubation device comprises an elongated imaging device, the method further comprising using the elongated imaging device to image vocal cords of the patient prior to pulling on the flexible tube.

43. The method of claim 40 wherein the tube retention section comprises a retention gap of less than an outside diameter of the flexible tube and greater than an outside diameter of a desired intermediate intubation device.

44. The method of claim 43, wherein, after the pulling, the shaft of the intermediate intubation device is caused to move through the retention gap and out of the tube retention section.

45. A method of intubating a patient, the method comprising:
providing an intubation system, the system comprising:
a first flexible tube;
a flexible tube holding device, the device comprising:
a handle; and
a blade coupled to the handle, the blade comprising a tube retention section sized to accept at least most of a cross-section of a tip portion of the first flexible tube and shaped to retain the tip portion, wherein the first flexible tube is detachably held against a back side of the intubating assisting device; and
an intermediate intubation device;
inserting the intubation system into an upper airway of the patient and maneuvering the intubation system to expand the upper airway;
visualizing the patient's vocal cords;
advancing a distal end of the intermediate intubation device between the vocal cords and into the patient's trachea based on the visualizing;
releasing the first flexible tube from the handle of the flexible tube holding device;
pulling on a proximal end of the first flexible tube to cause the first flexible tube to slide proximally out of the tube retention section while minimizing movement of the intermediate intubation device;
sliding a second flexible tube over the intermediate intubation device and into the trachea;
removing the intermediate intubation device;
removing the flexible tube holding device; and
leaving the second flexible tube within the trachea of the patient.

46. The method of claim 45, wherein the flexible tube holding device further comprises a quick-release closure mechanism coupled to the handle to hold the flexible tube at the back side of the handle, and wherein releasing the flexible tube comprises opening the quick-release closure.

47. The method of claim 45, wherein the handle comprises a groove along a length of a back side thereof, the groove sized to at least partially accept a desired flexible tube, wherein the blade comprises a proximal blade section coupled to the handle and having an opening along a back side thereof, wherein the blade further comprises a tube retention section coupled to the proximal blade section at an end thereof opposite the handle and along the opening, wherein the tube retention section is sized to accept at least most of a cross-section of a tip portion of the flexible tube and shaped to retain the tip portion, wherein the flexible tube is also detachably held in the groove, and wherein the releasing comprises releasing the flexible tube from the groove.

48. The method of claim 47, wherein the intermediate intubation device comprises an elongated imaging device, the method further comprising positioning the elongated imaging device to image the vocal cords of the patient prior to pulling on the first flexible tube.

49. The method of claim 45, wherein the intermediate intubation device comprises an elongated imaging device, wherein the visualizing is performed using the elongated imaging device, and wherein the pulling causes the first flexible tube to slide proximally over a shaft of the elongated imaging device and out of the tube retention section.

50. The method of claim 45, wherein the first flexible tube and the second flexible tube are a same flexible tube, and wherein the intermediate intubation device is sized to fit within the flexible tube.

51. The method of claim 50, wherein the tube retention section comprises a retention gap of less than a diameter of the flexible tube and greater than a diameter of the intermediate intubation device.

52. The method of claim 45, wherein the visualizing comprises one of visualization with an elongated imaging device situated within the first flexible tube, an elongated imaging device not situated within the first flexible tube, indirect visualization by reflection, use of a rigid videoscope, use of a standard laryngoscope, use of a fiber optic bronchoscope situated outside the first flexible tube, and use of a flexible, elongated videoscopic device situated outside the first flexible tube.

53. The method of claim 45, further comprising removing the first flexible tube and the flexible tube holding device off a proximal end of the intermediate intubation device and out of the patient prior to sliding the second flexible tube.

54. The method of claim 45, wherein the tube retention section of the flexible tube holding device comprises a retention gap of less than an outside diameter of the first flexible tube and greater than an outside diameter of the intermediate intubation device, the method further comprising:
causing a shaft of the intermediate intubation device to move through the retention gap and out of the flexible tube holding device prior to sliding the second flexible tube; and
removing the flexible tube holding device from the airway of the patient.

55. The method of claim 45, wherein the intermediate intubation device comprises one of an exchange catheter, an intubating bougie, a hollow intubation catheter sized to accept an elongated imaging device, an elongated imaging device, and an elongated imaging device within a hollow intubation catheter.

56. The method of claim 55, wherein the elongated imaging device comprises one of a fiber optic bronchoscope and a flexible, elongated videoscope.

57. The method of claim 45, wherein the intermediate intubation device comprises an elongated imaging device, the method further comprising removing the first flexible tube and the flexible tube holding device after removing the elongated imaging device.

58. The method of claim 45, wherein inserting the intubation system comprises advancing the intermediate intubation device in parallel with the flexible tube holding device and detachably held first flexible tube.

59. The method of claim 45, wherein the first flexible tube and the second flexible tube are a same flexible tube.
